(19) United States
(12) Patent Application Publication
(10) Pub. No.: US 2023/0324822 A1
(43) Pub. Date: Oct. 12, 2023

(54) SOLID PHASE TRANSFERS OF DNA AND OTHER REAGENTS

(71) Applicant: North Carolina State University, Raleigh, NC (US)

(72) Inventors: Albert Jun Qi Keung, Cary, NC (US); James M. Tuck, Raleigh, NC (US); Kyle Tomek, Raleigh, NC (US)

(73) Assignee: North Carolina State University, Raleigh, NC (US)

(21) Appl. No.: 18/023,521

(22) PCT Filed: Aug. 25, 2021
(86) PCT No.: PCT/US2021/047582

§ 371 Date: Feb. 27, 2023

Related U.S. Application Data
(60) Provisional application No. 63/070,061, filed on Aug. 25, 2020.

(51) Int. Cl. G03G 9/087; C12P 19/34; G03G 15/08; G03G 15/00

(52) U.S. Cl. CPC G03G 9/08775; C12P 19/34; G03G 15/0865; G03G 15/6585

(57) ABSTRACT

Disclosed are methods and systems for solid phase transfer of a reagent to a substrate. In some embodiments, the method comprises providing at least a first composition comprising a solid phase, wherein the solid phase comprises a reagent; and dispensing a first sample from the first composition onto a first coordinate on a substrate, whereby the first reagent is transferred to the substrate from the solid phase.

### CROSS REFERENCE TO RELATED APPLICATIONS

This application claims benefit of and priority to U.S. Provisional Patent Application Ser. No. 63/070,061, filed Aug. 25, 2020, the disclosure of which is incorporated herein by reference in its entirety.

### GRANT STATEMENT

This invention was made with government support under grant numbers CNS1650148 and CNS1901324 awarded by the National Science Foundation. The government has certain rights in the invention.

### TECHNICAL FIELD

The presently disclosed subject matter relates in some embodiments to methods and systems for solid phase transfer of polynucleotides and other reagents, such as molecular biology reagents, such as for the purposes of assembling a biological molecule for data storage or for other purposes.

### BACKGROUND

An increasing number of societal applications can be addressed by advances in synthetic biology and bioengineering. In particular, compelling approaches include the use of biomolecules, biomolecular circuits, and cells in biomanufacturing and for diagnostic sensors. Furthermore, mixtures of biological components can be freeze dried onto solid substrates such as paper and retain their activities when reconstituted with water, enabling easy storage and distribution. As these mixtures become more complex, and as the application space expands, new methods to rapidly array biomolecules, inorganic chemicals, and cells will be of great importance.

One example application that will require orders of magnitude improvements in the speed and scale of arraying molecules is DNA-based information storage. The world's information is rapidly passing zettabyte levels (1021 bytes), well beyond the limits of current electronic storage technology. Intriguingly, the biological molecule DNA (deoxyribonucleic acid) has the potential to store zettabyte amounts of information in only a cubic centimeter volume. Furthermore, DNA requires only a small fraction of the energy to store information compared with the large cooling requirements of electronic storage media. However, to achieve extreme capacity DNA-based storage systems, new technologies will be required. In particular, new physical and computational technologies are needed to address challenges that arise specifically from the high density of DNA strands in an extreme scale storage system.

The global datasphere is rapidly surpassing the projected material, space, and energy limits of electronic storage technologies. Reinsel, D., Gantz, J. & Rydning, J. IDC White Pap. Spons. by Seagate 1-25 (2017). DNA features high raw capacity, long-term durability, and minimal energy usage, thus representing a transformative solution as an extreme-scale archival storage medium. While there are limitations centered around the economics of DNA synthesis and sequencing, costs are rapidly decreasing. Carlson, R. Synthesis.com 20-23 (2014). In six years, the field has transitioned from storing and sequencing a 0.69 MB book to a 200 MB database including a music video. Church, G. M., Gao, Y. & Kosuri, S. Science. 337, 1628-1628 (2012); Organick, L. et al. Nat. Biotechnol. 36, 242-249 (2018).

The “DNA Economy” is rapidly permeating wide-ranging industries including human and veterinary health, commodities production, bioremediation, and most recently digital information storage. This “economy” fundamentally relies on technologies to synthesize and sequence DNA. Additional approaches thus represent an ongoing need in the art.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, the presently disclosed subject matter provides a method for solid phase transfer of a reagent to a substrate. In some embodiments, the method comprises: (a) providing at least a first composition comprising a solid phase, wherein the solid phase comprises a first reagent; and (b) dispensing a first sample from the first composition onto a first coordinate on a substrate, whereby the first reagent is transferred to the substrate from the solid phase. In some embodiments, the transfer occurs by electrostatic transfer. In some embodiments, the solid phase comprises a powder, a bead or a combination thereof, wherein the first reagent is provided on the powder, the bead, or the combination thereof. In some embodiments, the substrate comprises a charge and/or comprises a reagent pre-loaded at one or more coordinates on the substrate. In some embodiments, the first composition comprises a positively charged moiety or a negatively charged moiety.

In some embodiments, the first reagent comprises a chemical entity selected from the group consisting of a polynucleotide, a polypeptide, a lipid, a small molecule organic chemical, a detectable moiety, an inorganic chemical entity, and/or a molecular hybrid of any of these groups, or a cell. In some embodiments, the polynucleotide component comprises a first nucleic acid oligomer block. In some embodiments, the first oligomer block comprises a codeword.

In some embodiments, step (b) comprises providing a laser printer comprising at least a first toner cartridge for the first composition, wherein the laser printer is configured to dispense the first sample from the first toner cartridge onto the coordinate on the substrate. In some embodiments, the method comprises providing at least a second composition comprising a solid phase, wherein the solid phase comprises a second reagent; and dispensing a second sample from the second composition onto a coordinate on a substrate, wherein the coordinate is the first coordinate or is a second, different coordinate, whereby the second reagent is transferred to the substrate from the solid phase. In some embodiments, the method comprises dispensing the first sample from the first composition onto a coordinate on the substrate and dispensing the second sample from the second composition onto the coordinate on the substrate, such that the first and second reagent are collocated on the substrate. In some embodiments, the method comprises providing a laser printer comprising at least a first toner cartridge for the first composition and a second toner cartridge for the second composition, wherein the laser printer is configured to dispense the first sample from the first toner cartridge onto the first coordinate on the substrate and to dispense a second sample from the second toner cartridge onto the first or the second coordinate on the substrate. In some embodiments, the method comprises providing a condition necessary to cause an interaction between the first and second reagent, such as a reaction between the first and second reagent, such as a reaction to physically link the first and second reagent. In some embodiments, the method comprises dispensing a reaction mix onto the first coordinate on the substrate to provide a condition necessary to cause an interaction between the first and second reagent, such as a reaction between the first and second reagent, such as a reaction to physically link the first and second reagent. In some embodiments, the reaction mix comprises a ligase, a polymerase, a recombinase, an exonuclease, a restriction endonuclease, a nickase, or any combination thereof. In some embodiments, the laser printer, or a system comprising the laser printer, comprises a component configured to provide a condition necessary to cause an interaction between the first and second reagent, such as a reaction between the first and second reagent, such as a reaction to physically link the first and second reagent. In some embodiments, the component comprises a third toner cartridge, an incubator, pin array, or any combination thereof.

In some embodiments, the solid phase comprises a powder, a bead or a combination thereof, wherein the first reagent and the second reagent are provided on the powder, the bead, or the combination thereof. In some embodiments, the substrate comprises a charge. In some embodiments, the first reagent and the second reagent each comprise a positively charged moiety or a negatively charged moiety. In some embodiments, the first reagent and the second reagent are the same or different, and/or each comprise a chemical entity selected from the group consisting of a polynucleotide, a polypeptide, a lipid, a small molecule organic chemical, a detectable moiety, an inorganic chemical entity, and/or a molecular hybrid of any of these groups. In some embodiments, the polynucleotide component comprises a first nucleic acid oligomer block and a second nucleic acid oligomer block. In some embodiments, the first oligomer block and the second oligomer each comprise a codeword. In some embodiments, the method comprises providing one or more additional compositions, each additional composition comprising an additional reagent, wherein each of the additional reagent can independently be the same or different from the first or the second reagent; dispensing a sample from each of the one or more additional compositions onto a coordinate on a substrate, wherein the coordinate is the first coordinate, is the second coordinate, or is one or more different coordinates, whereby the each of the additional reagents is transferred to the substrate from the solid phase. In some embodiments, the method comprises dispensing a sample from each of the one or more additional compositions onto a coordinate on the substrate, such that the first, second, and one or more additional reagents are collocated on the substrate; and optionally providing a condition necessary to cause an interaction between or among the first, second, and/or one or more additional reagents, such as a reaction between or among the first, second, and/or one or more additional reagents, such as a reaction to physically link the first, second, and/or one or more additional reagents. In some embodiments, the method comprises providing a laser printer comprising one or more additional toner cartridges for the one or more additional compositions, wherein the laser printer is configured to dispense a sample from each of the one or more additional toner cartridges onto a coordinate on the substrate, optionally wherein the first, second, and one or more additional reagents are collocated on the substrate.

In some embodiments, a system suitable for use in carrying out any of the presently disclosed methods is provided.

It is an object of the presently disclosed subject matter to provide methods and systems for solid phase transfer of polynucleotides and other reagents. This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description and Figures.

### BRIEF DESCRIPTION OF THE DRAWINGS

FIG. 1 is a schematic showing the unit processes of a generic DNA storage system.

FIGS. 2A-2D are schematic drawings showing representative one-pot enzyme-based (FIG. 2A) Golden-Gate and (FIG. 2C) Overlap Extension PCR DNA assembly methods that use complimentary overhang sequences on the ends of ‘codeword’ monomers to (FIG. 2B, FIG. 2D) assemble mixtures of DNA strands of different sizes as well as specific strands.

FIGS. 3A-3C are a schematic showing a process in accordance with the presently disclosed subject matter.

FIG. 4 is a photographic image of a toner cartridge for a printer used in a system in accordance with the presently disclosed subject matter.

FIG. 5 is an image of gel electrophoresis of a PCR analysis of a method in accordance with the presently disclosed subject matter.

FIG. 6 is an image of capillary electrophoresis of a PCR analysis of a method in accordance with the presently disclosed subject matter.

FIG. 7 is a set of images of gel electrophoresis and reaction tubes of a PCR analysis of a method in accordance with the presently disclosed subject matter.

FIG. 8 is an image of capillary electrophoresis analysis of a method in accordance with the presently disclosed subject matter.

FIG. 9 is a set of images of a petri dish employed in a method of the presently disclosed subject matter.

FIG. 10 is a schematic of a system that can be employed in accordance with the presently disclosed subject matter.

### DETAILED DESCRIPTION

The presently disclosed subject matter demonstrates in some embodiments that reagents, such as biomolecules, such as DNA molecules, can be transferred to a substrate in solid state form, such as by using an electrostatic approach. By way of example and not limitation, DNA-based data storage is used here as an example application; other applications include by are not limited to the printing of molecular components for synthetic biology circuits on paper that can be used as distributable sensors or bioproduction units, and printing of pharmaceuticals or reaction mixes in biomanufacturing processes.

Additional applications of the presently disclosed subject matter include but are not limited to:
Preserving the stability of biological proteins, nucleic acids, lipids, neurotransmitters, other compounds, that are typically less stable when reconstituted in a solvent. By being able to transfer or dispense biological components as solids, this can preserve stability.
Printing or dispensing biological components onto a substrate like paper for paper-based genetic circuits or diagnostic sensors that detect the presence of metabolites/viruses/pathogens/RNAs/DNAs in a fluid, where the fluid is added to the paper that rehydrates the genetic circuit or sensor components and stimulates the genetic circuit or sensor to respond.
Solid phase handling of biological components can yield higher spatial resolution and faster speeds than liquid handling.
Solid phase handling of biological components can be useful in manufacturing, for example, in dispensing phosphoramidites or dNTPs in DNA synthesis or DNA assembly and for mixing compounds for a compound pharmaceutical.
Solid phase handling of biological components and chemicals could be useful in drug screening, for example where an in vitro biochemical assay is printed onto paper and arrays of drug compounds are transferred as well, or where cells like bacteria or yeast are dispensed on a substrate and drugs or chemicals are directly transferred onto the cells.
Solid phase handling of biological components could be used in bench-scale printers to print complex arrays of media or chemical compositions that a user, such as a graduate student, needs for experiments.
   In all of these cases, current methods include liquid handling robots that pipette or use acoustic transfer of liquids, or manual liquid pipetting, or pin-based transfer of small amounts of liquid are used to dispense reagents with no solid phase aspect.

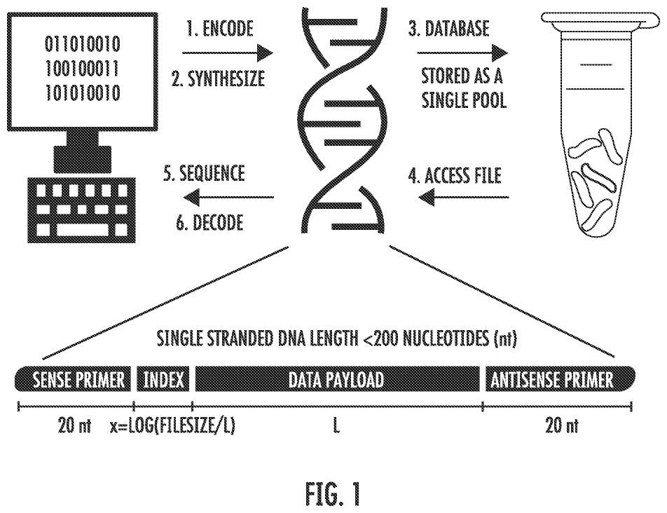

DNA is a natural biopolymer in which 0s and 1s can be stored as strings of A, C, T, and G nucleotides (nt). A generic end-to-end DNA storage system is shown in FIG. 1 in which digital information is (1) encoded into a set of unique DNA sequences, (2) synthesized and (3) stored as a pooled library of DNA strands, (4) physically accessed through polymerase chain reaction (PCR) or other DNA homology-based reactions, (5) read by DNA sequencing, and finally (6) decoded back into digital form. While DNA storage is an early stage technology, in fact each of these ‘unit processes’ have already been achieved^{1-12.} The presently disclosed subject matter addresses at least step (2), scaling the synthesis or ‘writing’ of data into DNA molecules, to provide improved end-to-end DNA storage methods and systems.

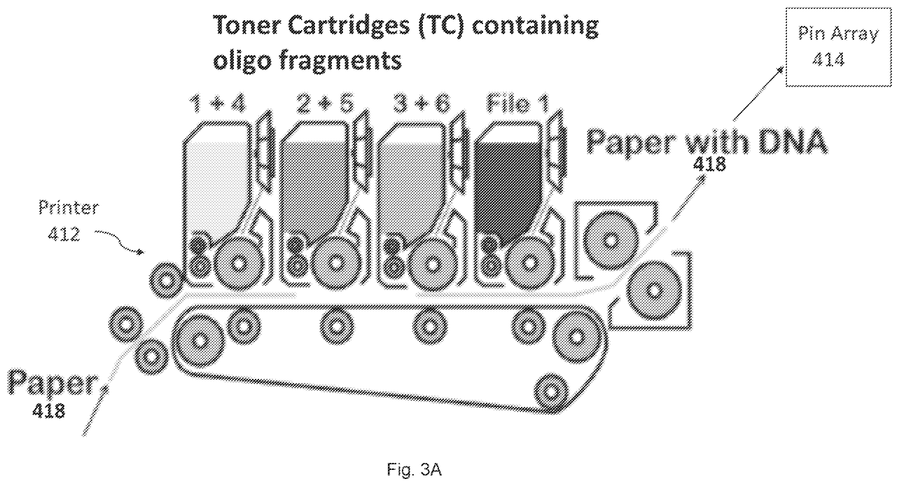

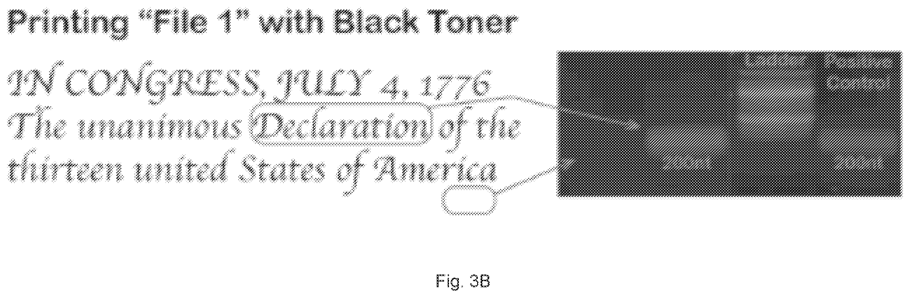

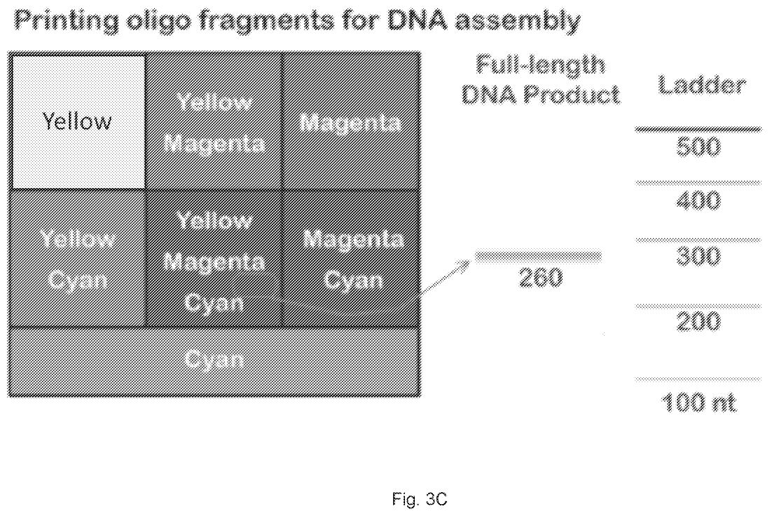

By way of example and not limitation, the presently disclosed subject matter pertains in some embodiments to methods and systems for incorporating of DNA into the toner of a laser printer, using the printer to print many spots onto a disposable or reusable paper or other sheet-like substrate, and then repeating with many other toners. In some embodiments, each toner cartridge contains a different monomer building block (a DNA oligomer), much like CMYK or RGB toner cartridges contain different ink colors. Then, different DNA strands are assembled by printing different sets of monomers onto each spot on the substrate. See FIGS. 3A-3C. The spots are rehydrated with a reaction mix, optionally a solution, containing enzymes, ligases, and/or polymerases, with suitable supporting components such as buffers and the like, to assemble the DNA blocks together. Then the assembled DNA is washed from the substrate and purified. It is believed that a laser-printer based system provides a substantially faster and cheaper alternative to inkjet-based printing systems described in the art. The presently disclosed subject matter also demonstrates that long strands of DNA can be self-assembled from smaller blocks of DNA, which can be manufactured cheaply at scale. Together, these innovations provide the foundation for a highly scalable DNA “printing press” that leverages the economies of scale of enzymatic DNA self-assembly and the speed, precision, and scalable continuous operation of laser printing. Furthermore, laser printing itself is a mature technology that can be scaled industrially.

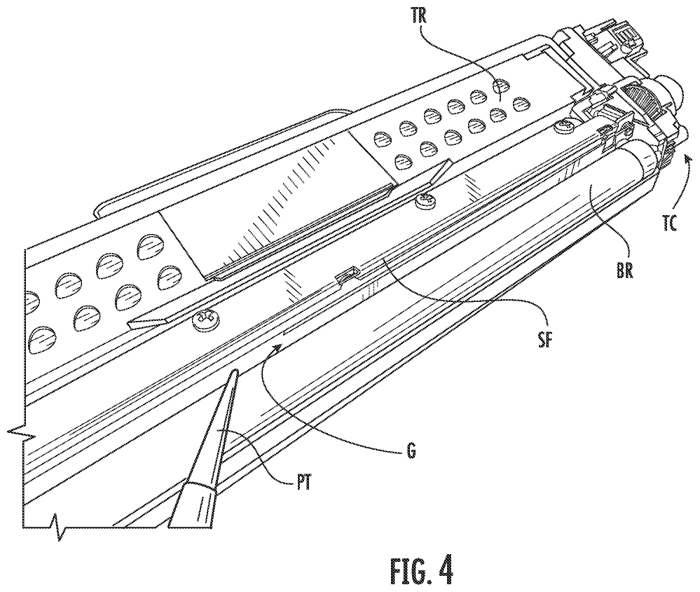

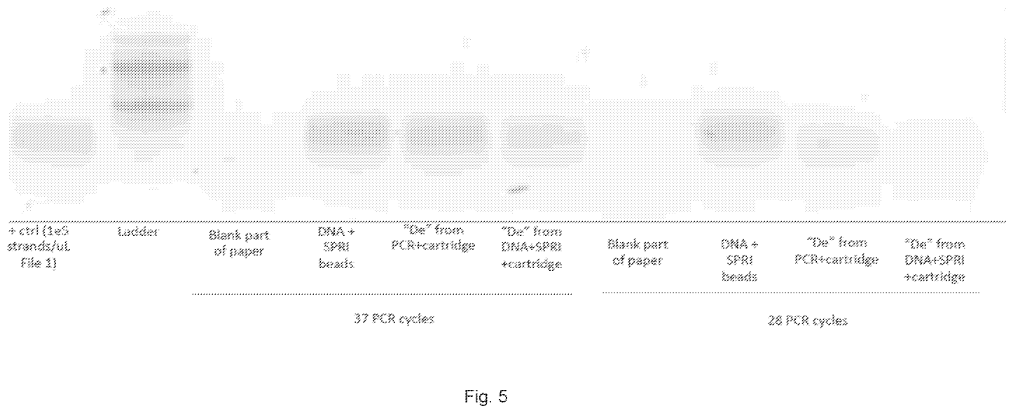

Thus, an aspect of the presently disclosed subject matter is to place appropriate polynucleotide (e.g., DNA) building blocks to a correct location as quickly as possible for assembly by electrostatic transfer and/or by printing with laser printer. Polynucleotide, e.g. DNA, building blocks are collocated for assembly reactions using laser printing. Additional aspects provided by the presently disclosed subject include but are not limited to high speed and high resolution/precision, small reaction volumes, and nucleic acid material (e.g., DNA) stored in highest density, powdered form. In some embodiments, each component reagent (e.g., nucleic acid) gets its own roller/cartridge. In some embodiments, the toner/powder composition is engineered for precise printing and nucleic acid material (e.g., DNA) stability. In some embodiments, the printer is engineered to contain many/multiple cartridges, such as but not limited to 256. In FIGS. 4 and 5, discussed below, it is shown that the presently disclosed subject matter can print and perform PCR and ligation enzymatic reactions on a file and can print multiple nucleic acids (e.g., DNAs) to the same location and assemble into a full strand. In using a laser printer, a laser induces change in electrostatics in a spatial pattern on a drum/roller, and the toner is transferred by the electrostatics. However, other solid phase transfer approaches as would be apparent to one of ordinary skill in the art upon a review of the instant disclosure can be used in accordance with the presently disclosed subject matter and fall within the scope of the presently disclosed subject matter, including other electrostatic transfer based approaches.

In some embodiments, the presently disclosed subject matter adapts the chemical make-up of a reagent (e.g., DNA) “toner” composition for electrostatic transfer. The presently disclosed subject matter provides that DNA can be electrostatically transferred and/or laser printed onto paper. In some embodiments, the composition includes an electrostatic charge for efficient and highly controlled DNA transfer. In some embodiments, the toner composition carries the DNA from the toner cartridge to the paper. In some embodiments, different substrates with tailored properties for DNA information storage, as compared to traditional office paper, are provided.

In some embodiments, the presently disclosed subject matter provides for optimization of a chemical composition of DNA “toner” for electrostatic transfer in laser printing to further facilitate the molecular assembly of DNA through fundamental chemistry for cost-effective writing of information into the 10¹¹ or greater numbers of strands of DNA necessary for terabyte (TB) and greater storage. That is, in some embodiments, the presently disclosed subject matter provides for optimization of a chemical composition of DNA “toner” for electrostatic transfer in laser printing to further facilitate physically executing each of these >10¹¹ reactions. While these reactions can easily be performed in parallel, it involves delivering the proper set of DNA codewords for each distinct strand to the right places in some form of high-density reaction array. Current approaches have relied on different forms of liquid handling including microfluidics and inkjet printing^{27,28}. However, liquid handling poses considerable challenges in achieving the necessary scaling and speed even when using automatic high throughput acoustic liquid handlers like the LabCyte Echo that would take ˜24 hours to print 1 GB of data³⁰⁻³⁵.

In some aspects, the presently disclosed subject matter provides for a transition from inkjet printers to much faster laser printers. Laser printing is fast, has high spatial resolution, does not involve liquid handling, and could be integrated into a continuous process. Furthermore, there is an additional advantage of providing more stable storage of DNA codewords in solid phase form as a DNA “toner” as opposed to liquid storage which is more prone to DNA hydrolysis over time³⁵⁻³⁵.

Representative embodiments of the presently disclosed subject matter are illustrated in FIGS. 3A-3C, where a series of different toner cartridges, each containing different DNA codewords or oligomers, print onto a paper substrate. In some embodiments, all oligomers needed to synthesize each DNA strand are printed from the appropriate toner cartridges onto the same spot on the paper. This approach provides a large number of distinct DNA strands to be assembled, one per “ink spot” on the paper. As shown in FIGS. 3A-3C and 4, using a simple home laser printer, it was possible print DNA onto standard office paper and perform enzymatic reactions on this DNA after printing (FIG. 3B). Thus, the presently disclosed subject matter provides methods and systems for laser printing/electrostatic transfer that address DNA toner composition, electrostatic charge requirements, and paper type. As laser printing of conventional ink is a mature industry and the hardware for optics and paper handling are mature technologies, in some embodiments, hardware technologies known the art are applied to DNA-based toners and electrostatic transfer. In some embodiments, the presently disclosed subject matter provides large-format reusable paper substrates that continuously roll through industrial-scale laser printing systems, with DNA codeword blocks printed onto arrayed spots and assembled enzymatically, and the substrate is reused and passed back again through the printing system.

### I. Definitions

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art.

While the following terms are believed to be well understood by one of ordinary skill in the art, the following definitions are set forth to facilitate explanation of the presently disclosed subject matter.

As used herein and in the appended claims, the singular forms “a,” “an,” and “the” include plural referents unless the context clearly dictates otherwise. For example, a nucleic acid molecule refers to one or more nucleic acid molecules. As such, the terms “a”, “an”, “one or more” and “at least one” can be used interchangeably. Similarly, the terms “comprising”, “including” and “having” can be used interchangeably. It is further noted that the claims may be drafted to exclude any optional element. As such, this statement is intended to serve as antecedent basis for use of such exclusive terminology as “solely,” “only” and the like, in connection with the recitation of claim elements, or use of a “negative” limitation.

Ranges can be expressed herein as from “about” one particular value, and/or to “about” another particular value. When such a range is expressed, some embodiments includes from the one particular value and/or to the other particular value. Similarly, when values are expressed as approximations, by use of the antecedent “about,” it will be understood that the particular value forms an embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed, then “less than or equal to 10” as well as “greater than or equal to 10” are also disclosed. It is also understood that the throughout the application, data are provided in a number of different formats, and that these data represent in some embodiments endpoints and starting points and in some embodiments ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The term “and/or”, when used in the context of a list of entities, refers to the entities being present singly or in combination.

The terms “optional” and “optionally” as used herein indicate that the subsequently described event, circumstance, element, and/or method step may or may not occur and/or be present, and that the description includes instances where said event, circumstance, element, or method step occurs and/or is present as well as instances where it does not.

The terms “assemble” and grammatically variations thereof are meant to encompass any synthesis employing reagents, including polynucleotide synthesis.

As used herein, the terms “complement,” “complementary,” “complementarity,” and the like, refer to the capacity for precise pairing between nucleobases in an oligonucleotide primer and nucleobases in a target sequence. Thus, if a nucleobase (e.g., adenine) at a certain position of an oligonucleotide primer is capable of hydrogen bonding with a nucleobase (e.g., thymidine, uracil) at a certain position in a target sequence in a target nucleic acid, then the position of hydrogen bonding between the oligonucleotide primer and the target nucleic acid is considered to be a complementary position. Usually, the terms complement, complementary, complementarity, and the like, are viewed in the context of a comparison between a defined number of contiguous nucleotides in a first nucleic acid molecule (e.g., an oligonucleotide primer) and a similar number of contiguous nucleotides in a second nucleic acid molecule (e.g., a DNA molecule bearing a data file in a database), rather than in a single base to base manner. For example, if an oligonucleotide primer is 25 nucleotides in length, its complementarity with a target sequence is usually determined by comparing the sequence of the entire oligonucleotide primer, or a defined portion thereof, with a number of contiguous nucleotides in a target molecule. An oligonucleotide primer and a target sequence are complementary to each other when a sufficient number of corresponding positions in each molecule are occupied by nucleobases which can hydrogen bond with each other. Positions are corresponding when the bases occupying the positions are spatially arranged such that, if complementary, the bases form hydrogen bonds. As an example, when comparing the sequence of an oligonucleotide primer to a similarly sized sequence in a target sequence, the first nucleotide in the oligonucleotide primer is compared with a chosen nucleotide at the start of the target sequence. The second nucleotide in the oligonucleotide primer (3′ to the first nucleotide) is then compared with the nucleotide directly 3′ to the chosen start nucleotide. This process is then continued with each nucleotide along the length of the oligonucleotide primer. Thus, the terms “specifically hybridizable”, “selectively hybridizable”, and “complementary” are terms which are used to indicate a sufficient degree of precise pairing or complementarity over a sufficient number of contiguous nucleobases such that stable and specific binding occurs between the oligonucleotide primer and a target nucleic acid.

Hybridization conditions under which a first nucleic acid molecule will specifically hybridize with a second nucleic acid molecule are commonly referred to in the art as stringent hybridization conditions. It is understood by those skilled in the art that stringent hybridization conditions are sequence-dependent and can be different in different circumstances. Thus, stringent conditions under which an oligonucleotide primer of this disclosure specifically hybridizes to a target sequence are determined by the complementarity of the oligonucleotide primer sequence and the target sequence and the nature of the assays in which they are being investigated. Upon a review of the instant disclosure, persons skilled in the relevant art are capable of designing complementary sequences that specifically hybridize to a particular target sequence for a given assay or a given use. Particular variations of hybridization conditions can be modified in accordance with aspects of the presently disclosed subject matter for accessing data files.

Once a target sequence has been identified, the oligonucleotide primer is designed to include a nucleobase sequence sufficiently complementary to the target sequence so that the oligonucleotide primer specifically hybridizes to the target nucleic acid. More specifically, the nucleotide sequence of the oligonucleotide primer is designed so that it contains a region of contiguous nucleotides sufficiently complementary to the target sequence so that the oligonucleotide primer specifically hybridizes to the target nucleic acid. Such a region of contiguous, complementary nucleotides in the oligonucleotide primer can be referred to as an “antisense sequence” or a “targeting sequence.”

It is well known in the art that the greater the degree of complementarity between two nucleic acid sequences, the stronger and more specific is the hybridization interaction. It is also well understood that the strongest and most specific hybridization occurs between two nucleic acid molecules that are fully complementary. As used herein, the term fully complementary refers to a situation when each nucleobase in a nucleic acid sequence is capable of hydrogen binding with the nucleobase in the corresponding position in a second nucleic acid molecule. In some embodiments, the targeting sequence is fully complementary to the target sequence. In some embodiments, the targeting sequence comprises an at least 6 contiguous nucleobase region that is fully complementary to an at least 6 contiguous nucleobase region in the target sequence. In some embodiments, the targeting sequence comprises an at least 8 contiguous nucleobase sequence that is fully complementary to an at least 8 contiguous nucleobase sequence in the target sequence. In some embodiments, the targeting sequence comprises an at least 10 contiguous nucleobase sequence that is fully complementary to an at least 10 contiguous nucleobase sequence in the target sequence. In some embodiments, the targeting sequence comprises an at least 12 contiguous nucleobase sequence that is fully complementary to an at least 12 contiguous nucleobase sequence in the target sequence. In some embodiments, the targeting sequence comprises an at least 14 contiguous nucleobase sequence that is fully complementary to an at least 14 contiguous nucleobase sequence in the target sequence. In some embodiments, the targeting sequence comprises an at least 16 contiguous nucleobase sequence that is fully complementary to an at least 16 contiguous nucleobase sequence in the target sequence. In some embodiments, the targeting sequence comprises an at least 18 contiguous nucleobase sequence that is fully complementary to an at least 18 contiguous nucleobase sequence in the target sequence. In some embodiments, the targeting sequence comprises an at least 20 contiguous nucleobase sequence that is fully complementary to an at least 20 contiguous nucleobase sequence in the target sequence.

It will be understood by those skilled in the art that the targeting sequence may make up the entirety of an oligonucleotide primer of this disclosure, or it may make up just a portion of an oligonucleotide primer of this disclosure. For example, in an oligonucleotide primer consisting of 30 nucleotides, all 30 nucleotides can be complementary to a 30 contiguous nucleotide target sequence. Alternatively, for example, only 20 contiguous nucleotides in the oligonucleotide primer may be complementary to a 20-contiguous nucleotide target sequence, with the remaining 10 nucleotides in the oligonucleotide primer being mismatched to nucleotides outside of the target sequence. In some embodiments, oligonucleotide primers of this disclosure have a targeting sequence of at least 10 nucleobases, at least 11 nucleobases, at least 12 nucleobases, at least 13 nucleobases, at least 14 nucleobases, at least 15 nucleobases, at least 16 nucleobases, at least 17 nucleobases, at least 18 nucleobases, at least 19 nucleobases, at least 20 nucleobases, at least 21 nucleobases, at least 22 nucleobases, at least 23 nucleobases, at least 24 nucleobases, at least nucleobases, at least 26 nucleobases, at least 27 nucleobases, at least 28 nucleobases, at least 29 nucleobases, or at least 30 nucleobases in length.

In accordance with some embodiments of the presently disclosed subject matter, the inclusion of mismatches between a targeting sequence and a target sequence is possible without eliminating the functionality of the oligonucleotide primer. Moreover, such mismatches can occur anywhere within the interaction between the targeting sequence and the target sequence, so long as the oligonucleotide primer is capable of specifically hybridizing to the targeted nucleic acid molecule. Thus, oligonucleotide primers of this disclosure may comprise up to about 50% nucleotides that are mismatched, thereby disrupting base pairing of the oligonucleotide primer to a target sequence, as long as the oligonucleotide primer specifically hybridizes to the target sequence. In some embodiments, oligonucleotide primers comprise no more than 50%, no more than 45%, no more than 40%, no more than 35%, no more than 30%, no more than 25%, no more than 20%, no more than about 15%, no more than about 10%, no more than about 5% or not more than about 3% of mismatches, or less. In some embodiments, there are no mismatches between nucleotides in the oligonucleotide primer involved in pairing and a complementary target sequence. In some embodiments, mismatches do not occur at contiguous positions. For example, in an oligonucleotide primer containing 3 mismatch positions, in some embodiments the mismatched positions can be separated by runs (e.g., 3, 4, 5, etc.) of contiguous nucleotides that are complementary with 15 nucleotides in the target sequence.

The use of percent identity is a common way of defining the number of mismatches between two nucleic acid sequences. For example, two sequences having the same nucleobase pairing capacity would be considered 100% identical. Moreover, it should be understood that both uracil and thymidine will bind with adenine. Consequently, two molecules that are otherwise identical in sequence would be considered identical, even if one had uracil at position x and the other had a thymidine at corresponding position x. Percent identity may be calculated over the entire length of the oligomeric compound, or over just a portion of an oligonucleotide primer. For example, the percent identity of a targeting sequence to a target sequence can be calculated to determine the capacity of an oligonucleotide primer comprising the targeting sequence to bind to a nucleic acid molecule comprising the target sequence. In some embodiments, the targeting sequence is at least 80% identical, at least 85% identical, at least 90% identical, at least 95% identical, at least 97% identical, at least 98% identical or at least 99% identical over its entire length to a target sequence in a target nucleic acid molecule. In some embodiments, the targeting sequence is identical over its entire length to a target sequence in a target nucleic acid molecule. It is understood by those skilled in the art that an oligonucleotide primer need not be identical to the oligonucleotide primer sequences disclosed herein to function similarly to the oligonucleotide primers described herein. Shortened versions of oligonucleotide primers taught herein, or non-identical versions of the oligonucleotide primers taught herein, fall within the scope of this disclosure. Non-identical versions are those wherein each base does not have 100% identity with the oligonucleotide primers disclosed herein. Alternatively, a non-identical version can include at least one base replaced with a different base with different pairing activity (e.g., G can be replaced by C, A, or T). Percent identity is calculated according to the number of bases that have identical base pairing corresponding to the oligonucleotide primer to which it is being compared. The non-identical bases may be adjacent to each other, dispersed throughout the oligonucleotide primer, or both. For example, a 16-mer having the same sequence as nucleobases 2-17 of a 20-mer is 80% identical to the 20-mer. Alternatively, a 20-mer containing four nucleobases not identical to the 20-mer is also 80% identical to the 20-mer. A 14-mer having the same sequence as nucleobases 1-14 of an 18-mer is 78% identical to the 18-mer. Such calculations are well within the ability of those skilled in the art. Thus, oligonucleotide primers of this disclosure comprise oligonucleotide sequences at least 80% identical, at least 85% identical, at least 90% identical, at least 92% identical, at least 94% identical at least 96% identical or at least 98% identical to sequences disclosed herein, as long as the oligonucleotide primers are able to bind and/or amplify a given target sequence.

The term “ligase” or “ligase enzyme,” as used herein, generally refers to any enzyme capable of catalyzing a ligase reaction, i.e., enzymes which catalyze the formation of a bond. Various ligases may be used for ligation. The ligases can be naturally occurring or synthesized. Examples of ligases include T4 DNA Ligase, T7 DNA Ligase, 13 DNA Ligase, Taq DNA Ligase, 9oNTM DNA Ligase, E. coli DNA Ligase, and SplintR DNA Ligase. Different ligases may be stable and function optimally at different temperatures. For example, Taq DNA Ligase is thermostable and T4 DNA Ligase is not. Moreover, different ligases have different properties. For example, T4 DNA Ligase may ligate bluntended dsDNA while T7 DNA Ligase may not.

The term “polymerase” or “polymerase enzyme,” as used herein, generally refers to any enzyme capable of catalyzing a polymerase reaction. Examples of polymerases include, without limitation, a nucleic acid polymerase. The polymerase can be naturally occurring or synthesized. An example polymerase is a Φ29 polymerase or derivative thereof. In some cases, a ligase is used (i.e., enzymes which catalyze the formation of a bond) in conjunction with polymerases or as an alternative to polymerases to construct new nucleic acid sequences. Examples of polymerases include a DNA polymerase, a RNA polymerase, a thermostable polymerase, a wild-type polymerase, a modified polymerase, E. coli DNA polymerase I, T7 DNA polymerase, bacteriophage T4 DNA polymerase, Φ29 DNA polymerase, Taq polymerase, Tth polymerase, Tli polymerase, Pfu polymerase Pwo polymerase, VENT polymerase, DEEPVENT polymerase, Ex-Taq polymerase, LA-Taw polymerase, Sso polymerase Poe polymerase, Pab polymerase, Mth polymerase ES4 polymerase, Tru polymerase, Tac polymerase, Tne polymerase, Tma polymerase, Tea polymerase, Tih polymerase, Tfi polymerase, Platinum Taq polymerases, Tbr polymerase, Tfl polymerase, Pfutubo polymerase, Pyrobest polymerase, KOD polymerase, Bst polymerase, Sac polymerase, Klenow fragment polymerase with 3′ to 5′ exonuclease activity, and variants, modified products and derivatives thereof.

Restriction enzymes can also be employed in accordance with the presently disclosed subject matter. Any suitable restriction enzyme (also referred to as restriction endonucleases) and suitable reaction conditions and reagents as would be apparent to one of ordinary skill in the art upon a review on the instant disclosure can be employed. Restriction endonucleases are available from many commercial sources, such as Thermo Fisher Scientific and Sigma Aldrich. By way of example and not limitation, Type I, Type II, Type III, and/or Type IV restriction enzymes can be employed. Additional specific no limiting examples include Asc1, EcoR1, HindIII, and/or XhoI restriction enzymes can be employed. Sticky ends may be created by digesting dsDNA with one or more endonucleases. Endonucleases (that may be referred to as restriction enzymes) may target specific sites (that may be referred to as restriction sites) on either or both ends of dsDNA molecule, and create a staggered cleavage (sometimes referred to as a digestion) thus leaving a sticky end. The digest may leave a palindromic overhang (an overhang with a sequence that is the reverse complement of itself). If so, then two components digested with the same endonuclease may form complimentary sticky ends along which they may be assembled with a ligase. The digestion and ligation may occur together in the same reaction if the endonuclease and ligase are compatible. The reaction may may be used to create components with sticky ends.

3′ exonucleases may be used to chew back the 3′ ends from dsDNA, thus creating 5′ overhangs. Likewise, 5′ exonucleases may be used to chew back the 5′ ends from dsDNA thus creating 3′ overhangs. Different exonucleases may have different properties. For example, exonucleases may differ in the direction of their nuclease activity (5′ to 3′ or 3′ to 5′), whether or not they act on ssDNA, whether they act on phosphorylated or non-phosphorylated 5′ ends, whether or not they are able to initiate on a nick, or whether or not they are able to initiate their activity on 5′ cavities, 3′ cavities, 5′ overhangs, or 3′ overhangs. Different types of exonucleases include Lambda exonuclease, RecJf, Exonuclease III, Exonuclease I, Exonuclease T, Exonuclease V, Exonuclease VIII Exonuclease VII, Nuclease BAL_31, T5 Exonuclease, and T7 Exonuclease.

Nickases are endonucleases that recognize a specific recognition sequence in double stranded DNA, and cut one strand at a specific location relative to said recognition sequence, thereby giving rise to single-stranded breaks in duplex DNA. Nickases include but are not limited to Nb.BsrDI, Nb.BsmI, Nt.BbvCI, Nb.BbvCI, Nb.BtsI and Nt.BstNBI. Use of a nickase on the double-stranded amplification product results in a single-stranded nick.

In some aspects, the presently disclosed subject matter provides reactions that support recombination between complementary recombination motifs. In some instances, recombination requires a recombination enzyme. A recombination enzyme may be a recombinase. A recombination enzyme may be an integrase. A recombination enzyme may be, e.g., a serine family recombinase or tyrosine family recombinase. The serine and tyrosine recombinase families are each named according to the conserved nucleophilic amino acid that interacts with DNA during recombination. Serine family recombinases include HIN invertase, which recognizes hix sites, and Tn3 resolvase. Tyrosine family recombinases included lambda integrase, which recognizes att sites, Cre, which recognizes lox sites, and FLP, which recognizes frt sites. Other recombination enzymes are known in the art.

Before the present compounds, compositions, articles, devices, and/or processes are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### II. Representative Embodiments of the Presently Disclosed Subject Matter

In some embodiments, the presently disclosed subject matter provides a method for solid phase transfer of a reagent, such as a biomolecule or biomolecule component, to a substrate. In some embodiments, the method comprises: (a) providing at least a first composition comprising a solid phase, wherein the solid phase comprises a first reagent; and (b) dispensing a first sample from the first composition onto a first coordinate on a substrate, whereby the first reagent is transferred to the substrate from the solid phase.

In some embodiments, the solid phase comprises a powder, a bead or a combination thereof. In some embodiments, the first reagent is provided on the powder, the bead, or on both a powder and a bead. Indeed, the first or any additional reagent as described herein can be provided on a powder, a bead, or on both a powder and a bead. The powder or bead can be prepared in accordance with any technique as would be apparent to one of ordinary skill in the art upon a review of the instant disclosure. By way of example and not limitation, powders can be generated through lyophilization, freeze-drying, or evaporation. By way of example and not limitation, beads could be generated through emulsion polymerization, chemical coatings or chemical vapor depositions.

In some embodiments, the solid phase transfer employs an electrostatic transfer. In some embodiments, the substrate comprises a charge, such as a charge deployed on a coordinate at which it is desired for the solid phase transfer to occur. In some embodiments, the first composition, the second composition, or an additional composition as described herein comprises a positively charged moiety or a negatively charged moiety. In some embodiments, the charge provided by one or more of the compositions is a polar opposite charge from the charged provided on the substrate. For example, the first reagent and the second reagent each comprise a positively charged moiety or a negatively charged moiety. In some embodiments, the substrate comprises a reagent pre-loaded at one or more coordinates on the substrate.

In some embodiments, reagents suitable for use in applications include by are not limited to the printing of molecular components for synthetic biology circuits on paper that can be used as distributable sensors or bioproduction units, and printing of pharmaceuticals or reaction mixes in biomanufacturing processes, are provided. In some embodiments, reagents include biological components for paper-based genetic circuits or diagnostic sensors that detect the presence of metabolites/viruses/pathogens/RNAs/DNAs in a fluid, where the fluid is added to the paper that rehydrates the genetic circuit or sensor components and stimulates the genetic circuit or sensor to respond, phosphoramidites or dNTPs in DNA synthesis or DNA assembly, reagents for mixing compounds for a compound pharmaceutical, reagents for drug screening, for example where an in vitro biochemical assay is printed onto paper and arrays of drug compounds are transferred as well, and/or cells like bacteria or yeast are dispensed on a substrate and drugs or chemicals are directly transferred onto the cells. In embodiments, the substrate can be provided with the cells (or indeed other reagents) already loaded onto at one or more coordinates. Alternatively, cells such as freeze-dried cells, can be delivered as the “first reagent” in the solid phase transfer, and drugs or chemicals are directly transferred onto the cells.

In some embodiments, the first reagent, a second reagent, and/or one or more additional reagents can be independently the same or different. In some embodiments, the first reagent, a second reagent, and/or one or more additional reagents each comprise a chemical entity selected from the group comprising a polynucleotide, a polypeptide, a lipid, a small molecule organic chemical, a detectable moiety, an inorganic chemical entity, and/or a molecular hybrid of any of the foregoing examples, or can comprise a cell.

In some embodiments, the polynucleotide reagent (first, second, and/or additional) comprises a nucleic acid oligomer block. In some embodiments, the oligomer block comprises a codeword. A reagent may be a distinct nucleic acid sequence. A reagent may be concatenated or assembled with one or more other reagents to generate other nucleic acid sequence or molecules. Other examples of reagents include codewords that are covalently linked to detectable moieties and/or non-organic molecules/chemicals. Examples of detectable moieties and/or non-organic molecules/chemicals include but are not limited to a quantum dot, such as a quantum dot that is comprises silicon or another inorganic element; a fluorescent dye (e.g., one or more organic or inorganic fluorescent dyes); or other molecules that confer some sort of handle for downstream handling or interfacing with electronics or nanopore sequencers. In some embodiments, the reagent can comprise an inorganic that could react to the end of one of the codewords. However, care is taken in choosing an inorganic reagent so that depositing an inorganic does not run afoul of the common laser printing of inorganic inks, etc.

In some embodiments, a laser printer comprising at least a first toner cartridge for the first composition is provided. In some embodiments, the laser printer is configured to dispense the first sample from the first toner cartridge onto the coordinate on the substrate.

In some embodiments, the method comprises providing at least a second composition comprising a solid phase, wherein the solid phase comprises a second reagent. The second reagent can be the same as or different from the first reagent. The second sample from the second composition is dispensed onto a coordinate on a substrate, whereby the second reagent is transferred to the substrate from the solid phase. In some embodiments, the coordinate is the first coordinate or is a second, different coordinate. In some embodiments, the method comprises dispensing the first sample from the first composition onto a coordinate on the substrate and dispensing the second sample from the second composition onto the coordinate on the substrate, such that the first and second reagents are collocated on the substrate.

In some embodiments, a laser printer is employed for electrostatic transfer. In some embodiments, the laser printer comprises at least a first toner cartridge for the first composition and a second toner cartridge for the second composition, wherein the laser printer is configured to dispense the first sample from the first toner cartridge onto the first coordinate on the substrate and to dispense a second sample from the second toner cartridge onto the first coordinate or onto a second, different coordinate on the substrate.

In some embodiments, the method comprises providing a condition necessary to cause an interaction between the first and second reagent, such as a reaction between the first and second reagent, such as a reaction to physically link the first and second reagent. In some embodiments, the method comprises dispensing a reaction mix onto a coordinate on the substrate wherein both the first reagent and the second reagent are collocated, so as to provide a condition necessary to cause an interaction between the first and second reagent, such as a reaction between the first and second reagent, such as a reaction to physically link the first and second reagent. In some embodiments, the reaction mix comprises an enzyme, such as but not limited to a ligase, a polymerase, recombinase, exonuclease, restriction endonuclease, nickase, or any combination thereof, and/or can comprise a buffer, such an aqueous buffer solution, such as a buffer solution comprising suitable supporting components for a reaction; a solvent; other reaction fluid, and the like. Indeed, the reaction mix can comprise reagents (in addition to the first, second, or additional reagent disposed on the substrate) that can be used in any desired manipulation as described herein, such as but not limited to methods of assembly of codewords such as but not limited to Gibson, recombination mediated assembly, and the like. In some embodiments, the laser printer, or a system comprising the laser printer, comprises a component configured to provide a condition necessary to cause an interaction between the first and second reagent, such as a reaction between the first and second reagent, such as a reaction to physically link the first and second reagent. In some embodiments, the component comprises a third toner cartridge, an incubator, or both. In some embodiments, the DNA codeword blocks are printed using electrostatic transfer/laser printing. In some embodiments, these printed spots are hydrated by stamping the paper spots with a reaction mix, e.g. a buffer transferred through pin arrays. By way of elaboration and not limitation, and with reference to FIGS. 3A, 10 and 11, an assembly line type approach can be employed, wherein the substrate or paper 418 is laser printed/solid state deposition of reagents (at coordinates 420), and the print heads/toner catridges TC are lined up like an assembly line with printer 412. Then, the substrate/paper 418 rolls along through the printer 412 to a pin array/acoustic liquid deposition device 414, or like device hydrates coordinates 420 on the substrate/paper 418. The reactions are then carried out to generate assembled DNA strands.

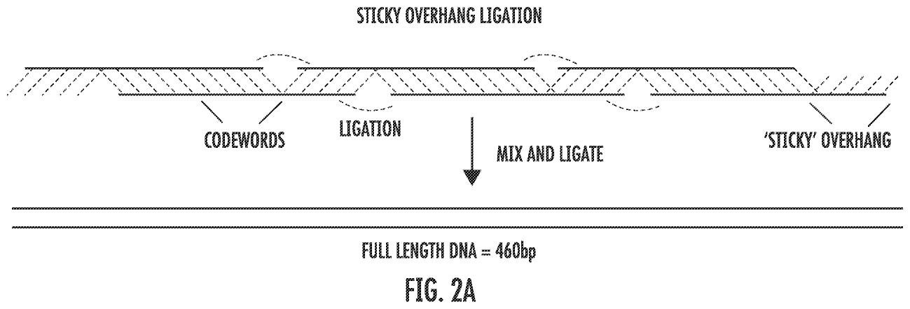

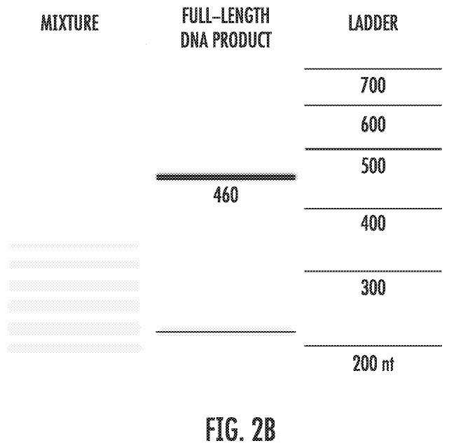

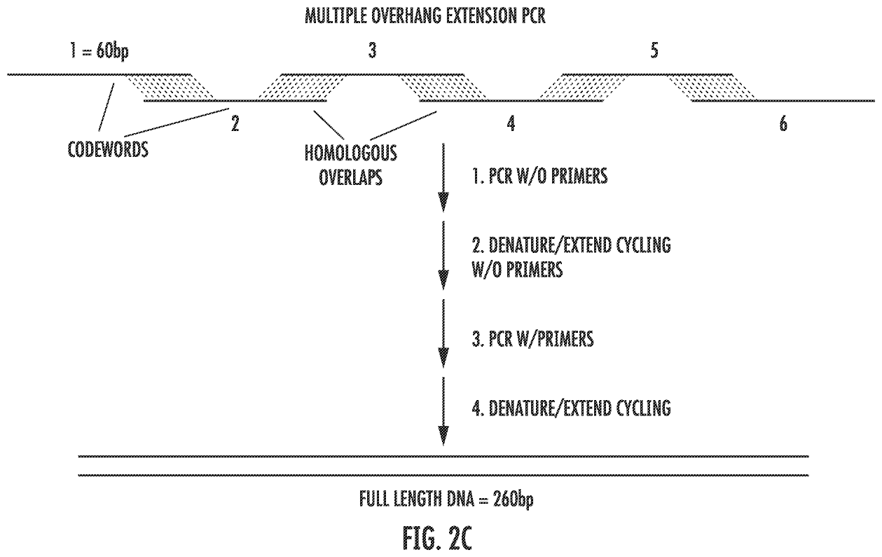

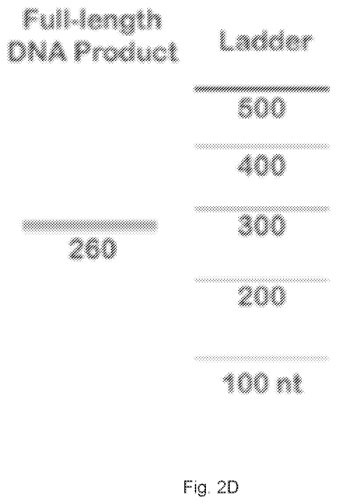

Any suitable method to assemble the first reagent, the second reagent, and/or more additional reagents as would be apparent to one of ordinary skill in the art upon a review of the instant disclosure can be employed. Exemplary assembly reactions are described herein and are also shown in FIGS. 2A and 2B. In some embodiments, conventional phosphoramidite DNA synthesis is employed. This process sequentially adds an A, C, T, or G, with each synthesis cycle involving the addition of a nucleotide, deprotection of the nucleotide for a subsequent nucleotide addition, and reagent washes and is typically employed in generating ˜200 nucleotide long DNA strands²¹⁻²³.

For DNA storage, it is not necessary to be able to create every arbitrary DNA sequence. Instead, blocks of nucleotides or “codewords” could be assembled together, with each codeword containing more information (e.g. a byte or more) than just an individual A, C, T, or G would. The advantage of this is at least twofold. First, codewords can be synthesized in bulk at considerable cost-savings. Second, the fields of molecular biology and synthetic biology have developed many high throughput one-pot DNA assembly methods including Golden Gate Assembly and Splice Overlap PCR where pieces of DNA have complementary overhangs that ligate to each other, without the need for multiple slow reaction cycles (FIG. 2A & 2C)²⁴⁻²⁶. The identity of the ligated overhangs dictates the sequence in which codewords are assembled. The ability to assemble monomer blocks that are each 25 nt long into 460 bp DNA strands, as well as create mixtures of DNA strands that are multiples of 25 nt, has been demonstrated (FIG. 2B & 2D).

Other representative assembly reaction conditions include but are not limited to the following ranges given for each individual reaction: 10¹-10¹² molecules of each DNA codeword block, 10¹-10¹⁰ molecules of ligase or polymerase enzyme, 1-1,000,000 nL volumes, 5-64800 second incubations, and 20 different buffer conditions that narrowly vary in ionic strength and pH around the standard buffer conditions of commercial ligase and polymerase buffers from New England Biolabs⁷⁷⁻⁸⁰.

In some embodiments, to mitigate the risk of each reaction generating a distribution of complete and incompletely assembled strands, low-cost paper-based size-exclusion chromatography or SPRI bead-based size separations are performed. In some embodiments, to mitigate the risk of high rates of errors in assembly, the number and sequence space of the overhangs are tuned and the overall length of the assembled products are reduced, and other one-pot assembly methods such as Gibson assembly, are employed^{24,26,82}. Short 50 bp read lengths can be performed to capture the possibility of incompletely assembled fragments.

In some embodiments, the method further comprises providing one or more additional compositions, each additional composition comprising an additional reagent. In some embodiments, each of the additional reagents can independently be the same or different from the first or the second reagent. In some embodiments, the method comprises dispensing a sample from each of the one or more additional compositions onto a coordinate on a substrate, wherein the coordinate is the first coordinate, is the second coordinate, or is one or more different coordinates, whereby the each of the additional reagents is transferred to the substrate from the solid phase. In some embodiments, the sample from each of the one or more additional compositions is dispensed onto a coordinate on the substrate, such that the first, second, and one or more additional reagents are collocated on the substrate. In some embodiments, the method comprises dispensing a sample from each of the one or more additional compositions onto a coordinate on the substrate, such that the first, second, and one or more additional reagents are collocated on the substrate; and optionally providing a condition necessary to cause an interaction between or among the first, second, and/or one or more additional reagents, such as a reaction between or among the first, second, and/or one or more additional reagents, such as a reaction to physically link the first, second, and/or one or more additional reagents.

In some embodiments, the laser printer comprises one or more additional toner cartridges for the one or more additional compositions. In some embodiments, the laser printer is configured to dispense a sample from each of the one or more additional toner cartridges onto a coordinate on the substrate. In some embodiments, the first, second, and one or more additional reagents are collocated on the substrate. In some embodiments, the first, second, and one or more additional reagents are located at different coordinates on the substrate.

Thus, an aspect of the presently disclosed subject matter is to place by solid phase transfer appropriate reagents or building blocks (e.g., biomolecules, such as a polynucleotide, such as DNA) at a correct location as quickly as possible for assembly. By way of example and not limitation, polynucleotide, e.g. DNA, building blocks are collocated for assembly reactions using laser printing. Additional aspects provided by the presently disclosed methods, such as by laser printing-based embodiments, include but are not limited to high speed and high resolution/precision, small reaction volumes, and nucleic acid material (e.g., DNA) stored in highest density, powdered form. In some embodiments, each reagent (e.g., component nucleic acid) gets its own roller/cartridge. In some embodiments, the toner/powder composition is engineered for precise printing and reagent material (e.g., DNA) stability. In some embodiments, the printer is engineered to contain many/multiple cartridges, such as but not limited to 256. In FIGS. 4 and 5, discussed below, it is shown that the presently disclosed subject matter can print and PCR (enzymatic reaction similar enough to ligation) a file and can print multiple nucleic acids (e.g., DNAs) to the same location and assemble into a full strand. In using a laser printer, a laser induces change in electrostatics in a spatial pattern on a drum/roller, and the toner is transferred by the electrostatics.

Robotic and acoustic liquid handling devices can quickly move and mix reagents. However, these liquid-handling devices only provide ˜1-2 orders of magnitude improvements in throughput over manual manipulations. Meanwhile there must be >10⁷ increases in throughput and speed when developing high-density DNA-based data storage systems. The presently disclosed subject matter provides that DNA can be electrostatically transferred and/or laser printed onto paper. In some embodiments, the composition includes an electrostatic charge for efficient and highly controlled DNA transfer. In some embodiments, the toner composition carries the DNA from the toner cartridge to the paper.

In some embodiments, electrostatic transfer is modified or enhaced. As shown in FIGS. 3A-3B, DNA can be deposited to and accessed from paper using an unmodified standard office laser printer. Laser printing works by inducing a positive charge on a roller or the paper substrate itself, while the toner is negatively charged. A laser is used to induce the positive charge in specific spatial patterns. An opposite configuration in which the roller/paper is negatively charged and the toner is positively charged is also used in some laser printers⁶⁹⁻⁷¹. Electrostatic transfer conditions are evaluated using an Electrostatic Charge Kit (Fisher #ELSTCH-01). Using this apparatus, it is shown herein that dry DNA mixed with KCl can achieve over 80% efficiency of transfer, the transfer can be spatially targeted, and the DNA is intact for downstream enzymatic reactions. Varying levels of electrostatic charge are imparted to a paper substrate, and DNA dried on a chargeless grounded surface are brought within 0.5 mm of the paper. The efficiency of transfer of the DNA are assessed by quantitative PCR. Thus, in some embodiments, different substrates having tailored properties for DNA information storage compared to traditional office paper are provided.

Different uses of paper-like printing substrates are also provided. In some embodiments, the paper substrate used for printing a reagent such as DNA is also used as a platform to execute the assembly reactions. Previous studies have shown that cellulose filter paper or quartz microfiber are appropriate surfaces for running enzymatic reactions^{75,76}. In both cases, the membranes can be preprocessed with blocking agents, for example 5% BSA or Tween-20, respectively, so that the reaction can efficiently run directly on the membrane. In some embodiments, these and other membranes are used as substrates both in the printing DNA and the assembling reactions. Reaction efficiencies are assessed. In some embodiments, a reusable membrane that can be recycled on a continuous printer “conveyor belt” is provided.

In some embodiments, the presently disclosed subject matter provides a formulation of DNA “toner”. In some embodiments, electrostatic transfer uses bare DNA. In some embodiments, other toner compositions are used. Compositions are provided, which comprise “biological toners” that include negatively charged compounds such as pectin (polygalacturonic acid), alginic acid, polyacrylic acid, sodium carboxymethyl cellulose, and positively charged compounds such as polyethylenimine, poly-L-lysine, DEAE-dextran, and PAMAM dendrimer that could serve as good carriers for negatively charged DNA⁷². In some embodiments, a positive to negative electrostatic transfer is employed and in some embodiments, the reverse is employed. Other potential additives include, TE buffer, LAB buffer. magnesium and potassium chloride, and calcium phosphate^{73,74}.

Further, the presently disclosed systems and methods can be used to prepare polynucleodtides for use in data storage systems. Thus, the presently disclosed subject matter can be used as part of storing and accessing digital data in polynucleotides (e.g., DNA) and can be used to address data storage issues in the DNA economy as described hereinabove. Representative techniques for storing and accessing data from DNA are known in the art and include those disclosed in International Publication No. WO 2020/096679, herein incorporated by reference in its entirety. Additionally, the presently disclosed systems and methods can be used for combinatorial assembly, such as combinatorial chemistry assembly, polynucleotide assembly, including combinatorial DNA assembly and gene synthesis. The combinatorial library can be used in various research efforts, such as but not limited to aptamer preparation, small molecule preparation, review of CRISPR technologies, and metabolic pathway studies.

In some embodiments, the presently disclosed subject matter provides a method for assembly of complete files. In some embodiments, the method comprises partitioning a digital file into small units that can be assembled separately as individual strands. This step can be accomplished any approach known in the art as would be apparent to one of ordinary skill in the art upon a review of the instant disclosure. One of the small units can be included in a first toner composition and thus can be a first component nucleic acid molecule in the first toner composition. Another small unit can be included in a second toner composition and thus can be a second component nucleic acid molecule in the second toner composition. Additional small units can likewise be included in additional toner compositions.

Each unit is mapped to a unique place on the “paper”, and the corresponding units are printed to assemble at that position. For example, the method can comprise dispensing a first sample from the first toner cartridge onto a coordinate on a substrate and dispensing a second sample from the second toner cartridge onto the coordinate on the substrate using a laser printer, such that the first and second component nucleic acid molecules are collocated on the substrate. In some embodiments, the method further comprises providing one or more additional toner compositions, each additional toner compositions comprising an additional component nucleic acid molecule; dispensing a sample from each of the one or more additional toner cartridges onto the coordinate on the substrate using the laser printer, such as by electrostatic transfer by the laser printer, such that the first, second, and one or more additional component nucleic acid molecules are collocated on the substrate. In some embodiments, the method comprises dispensing a sample from each of the one or more additional compositions onto a coordinate on the substrate, such that the first, second, and one or more additional reagents are collocated on the substrate; and optionally providing a condition necessary to cause an interaction between or among the first, second, and/or one or more additional reagents, such as a reaction between or among the first, second, and/or one or more additional reagents, such as a reaction to physically link the first, second, and/or one or more additional reagents. Thus, in some embodiments, the method comprises assembling all units in separate reactions. In some embodiments, after an initial assembly step the units belonging to a file are mixed together. Such embodiments may or may not include additional purification or amplification steps on each unit before mixing them with other units. In some embodiments, units may be mixed together to form larger assembled units or simply the aggregation of units needed to make an entire file. In some embodiments, an algorithm may determine which assembled units constitute a file and which should be mixed together. Thus, in some embodiments, the presently disclosed subject matter provides methods for laser printing and using it for making an entire file.

The presently disclosed subject matter pertains in some embodiments to methods and systems for incorporating of DNA into the toner of a laser printer, using the printer to print many spots onto a disposable or reusable paper or other sheet-like substrate, and then repeating with many other toners. In some embodiments, each toner cartridge contains a different monomer building block (a DNA oligomer, a codeword, and/or the like), much like CMYK or RGB toner cartridges contain different ink colors. Then, different DNA strands are assembled by printing different sets of monomers onto each spot on the substrate. See FIGS. 3A-3C. The spots are rehydrated with a reaction mix, such as a solution, containing enzymes, ligases, and/or polymerases to assemble the DNA blocks together. Then the assembled DNA is washed from the substrate and purified.

Continuing with reference to FIGS. 3A to 3C, DNA fragments were injected into the toner cartridges of a generic laser printer (FIG. 3A). The text of File 1, The Declaration of Independence, was printed in black toner. “Declaration” and a blank area were cut out and used as templates for a PCR reaction (circles and arrows in FIG. 3B). Only the black toner including File 1 produced a PCR product (circle and arrow in FIG. 3C). Referring to FIG. 3C, the color toners containing DNA fragments to be assembled were printed individually and in combination. Only when all 3 toners were present was the assembly reaction successful in producing the expected DNA fragment. PCR reactions are shown schematically in FIGS. 2A-2D and include multiple overhang extension PCR (MOEPCR).

Referring to FIG. 4, the end of a toner cartridge TC without the microchip was partially removed by unscrewing two screws, just to the extent that one end of the black roller BR could be lifted and pried outwards. A PCR product (without cleanup) was pipetted directly into the toner reservoir TR by lifting the black roller BR. Additional PCR product was pipetted into the gap G between the roller BR and what looks like a skimming flap SF (where the pipet tip PT is pointing to in the image of FIG. 4). The cartridge TC was reassembled and the toner was allowed to dry overnight, then the cartridge TC was shaken vigorously to mix the DNA and toner.

For DNA bound to SPRI beads, the DNA was bound to the beads, rinsed with 80% ethanol once, dried. About a gram of toner was collected from the reservoir TR by lifting the black roller BR out. This toner was mixed with the dried SPRI beads. This mixture was spread using a metal spatula directly onto the reservoir TR underneath the black roller BR as well as directly on the black roller BR.

Referring to FIG. 5, “The Declaration of Independence” in bold was printed repeatedly on the same piece of paper 8 times using the toner cartridge that had the PCR product directly added and dried. Then the SPRI beads were added to the toner and the same message was printed repeatedly 8 times on a fresh piece of paper.

Four samples were tested:
1) a blank part of the paper was cut out as a negative control
2) the SPRI beads mixed with the toner but not added to the cartridge.
3) The “De” was cut out after printing with the dried PCR product added to the cartridge
4) The “De” was cut out after printing with the SPRI beads added to the cartridge.

These samples were shredded with a razor and added to a 1.5 mL Eppendorf tube with 100 uL water, and heated to 95° C. for 5 minutes in the thermal mixer. The samples were then spun down at maximum speed ˜21,000 g, for 10 minutes. In the PCR workstation, PCRs were setup with 8.25 uL of each sample as the template, 1 uL pCl, 1 uL pNCl, 2.5 uL 10×PCR buffer w/oMgCl1, 0.75 MgCl1, 0.5 10 mM dNTP, 0.1 uL taq polymerase, 10 uL H₂O. A positive control was also included which was 1e5 strands/uL of File 1. One set of PCRs were performed to 37 cycles with 30 sec extension and 50° C. annealing temp. The other was performed to 28 cycles.

Referring to FIG. 5, no band was seen for the blank part of the paper was cut out as a negative control. Bands were seen at the expected location as compared to the positive control for the following: the DNA+SPRI beads mixed with the toner but not added to the cartridge; the “De” was cut out after printing with the dried PCR product added to the cartridge; and the “De” was cut out after printing with the SPRI beads added to the cartridge.

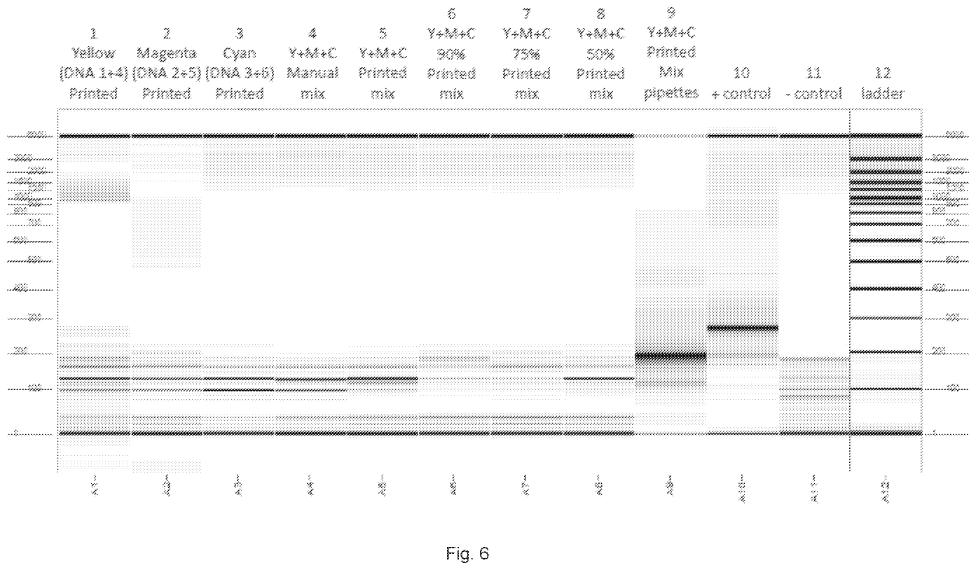

Referring to FIG. 6, the effect on standard toner ink on MOEPCR assembly reactions was assessed to facilitate toner composition design. DNA samples were added to standard toner catridges and printed on a piece of paper and the paper was purposefully jammed before the fuser could melt the toner. The yellow, magenta, and cyan toners contained DNA fragments 1+4, 2+5 and 3+6, respectively. The toner was printed, scraped into an eppendorf tube, and mixed with water as follows:
1—Yellow toner printed individually
2—Magenta toner printed individually
3—Cyan toner printed individually
4—Yellow, Magenta, and Cyan toners printed individually and mixed manually.
5—Yellow, Magenta, and Cyan toners printed to the same spot.
6—Yellow, Magenta, and Cyan toners printed to the same spot at 90% saturation.
7—Yellow, Magenta, and Cyan toners printed to the same spot at 75% saturation.
8—Yellow, Magenta, and Cyan toners printed to the same spot at 50% saturation.
9—Yellow, Magenta, and Cyan toners printed individually, the toner remaining on the pipette tips used for all scrapping added here.
10—Positive control sample with no toner
11—Negative control sample with no polymerase

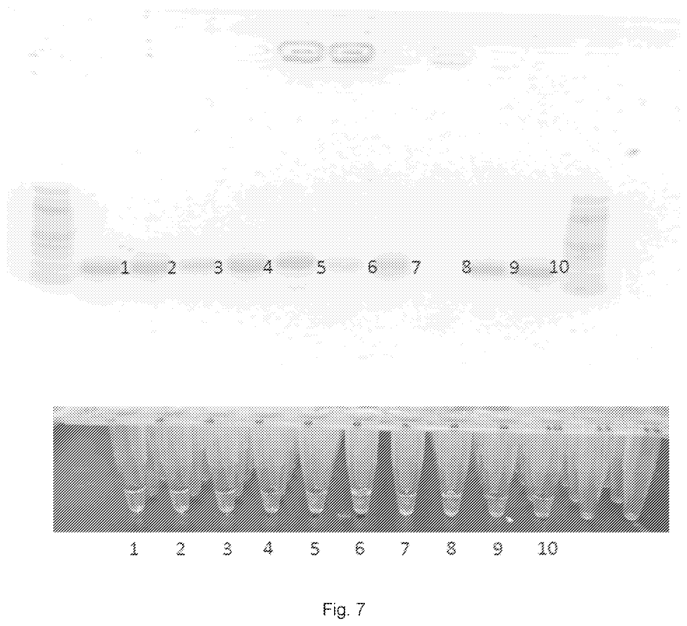

Referring to FIG. 7, the effect on standard toner ink on recovery reactions was assessed to facilitate toner composition design. DNA samples were mixed with each toner and reacted in a standard PCR. Toners were added to individual PCRs at low or high concentration as follows:
1—control (no toner)
2—control (no toner)
3—cyan low concentration (200 mg/mL)
4—cyan high concentration (50 mg/mL)
5—magenta low concentration (200 mg/mL)
6—magenta high concentration (50 mg/mL)
7—yellow low concentration (200 mg/mL)
8—yellow high concentration (50 mg/mL)
9—black low concentration (200 mg/mL)
10—black high concentration (50 mg/mL)

Magenta and yellow toners have inhibitory effect on PCR yield. This could explain problems with previous MOEPCRs. See also FIG. 6. To elaborate regarding the inhibition from the magenta, and yellow toners, PCR and other enzymatic reactions are sensitive reactions. Thus, the inhibitory observation was expected and thus the presently disclosed subject matter provides other components (such as KCl) to create biocompatible “toners”/carriers. The unexpected and surprising result was that the black toner shows no inhibition to the PCR reaction, which supports the presently disclosed approach to pursue printing via existing laser printer technology without much engineering while also providing “biocompatible” toners for particular use cases. Thus, in some embodiments, toners should be compatible with biological reactions and it is shown herein how some are and some are not. By way of example and not limitation, the black type toners are, and so are toners comprising biological compatible mixtures like KCl.

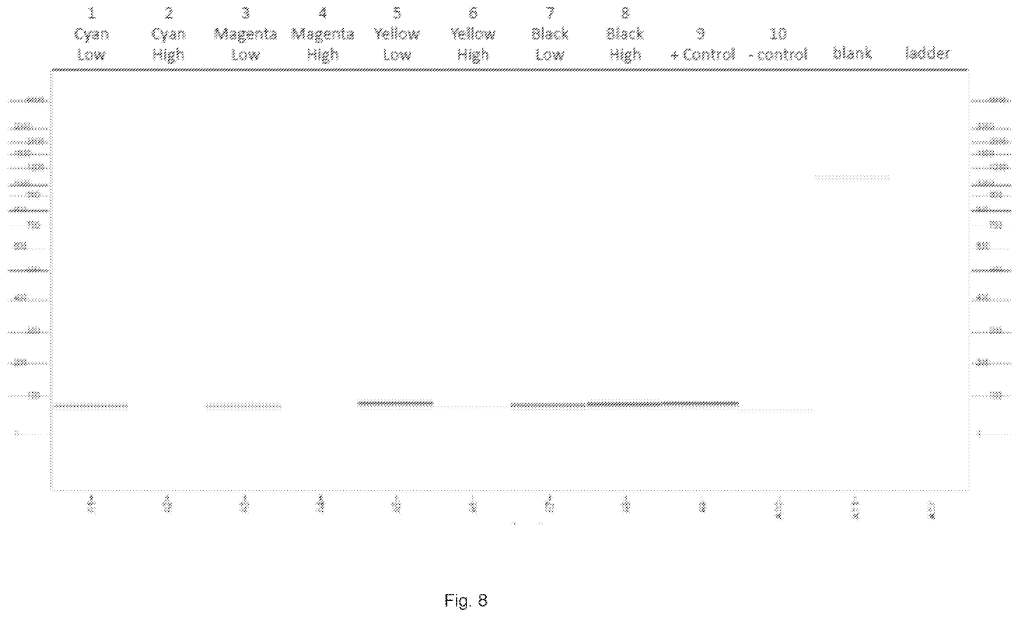

Referring to FIG. 8, the effect on standard toner ink on ligation reactions was assessed to facilitate toner composition design. DNA samples were mixed with each toner and reacted in a standard ligation. Toners were added to individual reactions at low or high concentration as follows:
1—cyan low concentration (200 mg/mL)
2—cyan high concentration (50 mg/mL)
3—magenta low concentration (200 mg/mL)
4—magenta high concentration (50 mg/mL)
5—yellow low concentration (200 mg/mL)
6—yellow high concentration (50 mg/mL)
7—black low concentration (200 mg/mL)
8—black high concentration (50 mg/mL)
9—positive control (no toner)
10—negative control (no ligase)

Cyan, magenta and yellow toners have inhibitory effect on ligation efficiency. To elaborate regarding the inhibition from the cyan, magenta, and yellow toners, ligation and other enzymatic reactions are sensitive reactions. Thus, the inhibitory observation was expected and thus the presently disclosed subject matter provides other components (such as KCl) to create biocompatible “toners”/carriers. The unexpected and surprising result was that the black toner shows no inhibition to the ligation reaction, which supports the presently disclosed approach to pursue printing via existing laser printer technology without much engineering while also providing “biocompatible” toners for particular use cases. Thus, in some embodiments, toners should be compatible with biological reactions and it is shown herein how some are and some are not. By way of example and not limitation, the black type toners are, and so are toners comprising biological compatible mixtures like KCl.

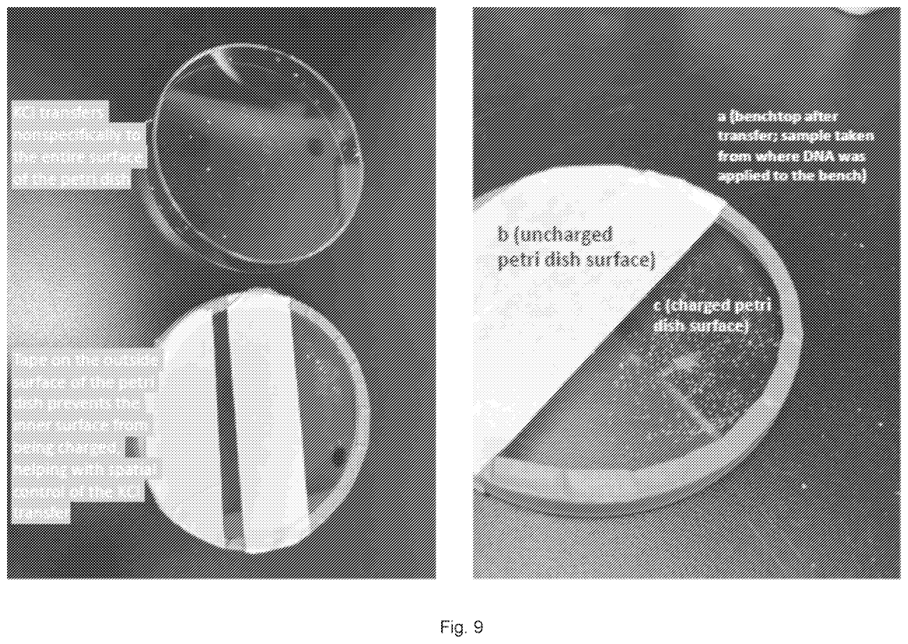

Referring to FIG. 9, potassium chloride (KCl) was used as a as “toner.” This experiment is designed to determine desirable, and in some embodiments optimal, electrostatic transfer conditions. An Electrostatic Charge Kit (Fisher #ELSTCH-01, Fisher Scientific) was employed. Using this apparatus, it was shown that dry DNA mixed with KCl can achieve over 80% efficiency of transfer, the transfer can be spatially targeted, and the DNA is intact for downstream enzymatic reactions.

Continuing with reference to FIG. 9, KCl transfers nonspecifically to the entire surface of the petri dish. Tape on the outside surface of the petri dish prevents the inner surface from being charged, helping with spatial control of the KCl transfer. At point (a) in FIG. 9, the benchtop after transfer is shown, wherein a sample was taken from where DNA was applied to the bench. FIG. 9 point (b) shows where a sample was taken from where the DNA was applied to the uncharged petri dish surface. FIG. 9, point (c) shows where a sample was taken charged petri dish surface. The following results were observed.

| Sample | DNA Strands remaining after transfer |
|---|---|
| a | 1.27 × 10¹³ strands (17.21%) |
| b | 1.03 × 10¹¹ strands (0.14%) |
| c | 6.1 × 10¹³ strands (82.65%) |

The following approach was then tried. The toner cartridge was emptied of normal toner and KCl was added. This trial was not successful since KCl did not transfer readily to paper. KCl is a neutral molecule and thus it appears that in at least some instances a neutral molecule would need to be charged prior to addition to a toner cartridge.

In some embodiments, the presently disclosed subject matter provides a substrate prepared in accordance the presently disclosed approaches. The substrate could freeze-dried but this is not necessary. As the substrate is solid and dry with no liquid, it can be vacuum sealed, stored under an inert gas, or even just put into a plastic bag or other storage container at room temperature without any special storage conditions.

### III. Systems of the Presently Disclosed Subject Matter

Disclosed herein in accordance with some embodiments of the presently disclosed subject matter are systems suitable for use in carrying out any of the processes set forth elsewhere herein. For example, several systems are disclosed in the Figures.

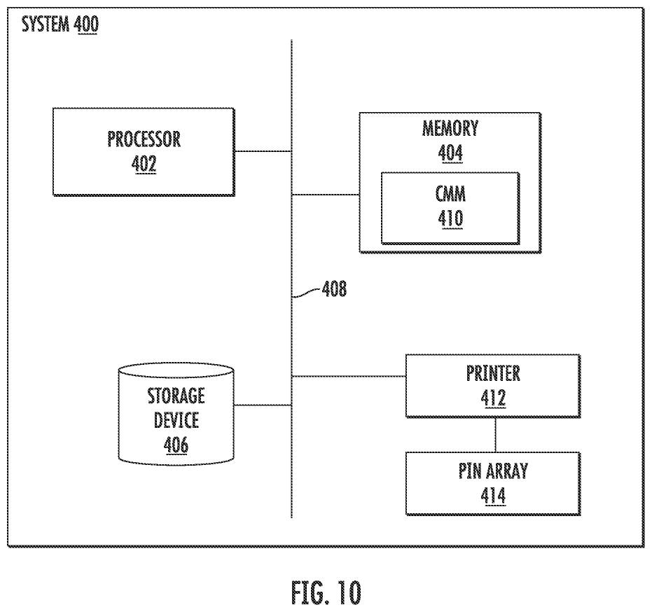

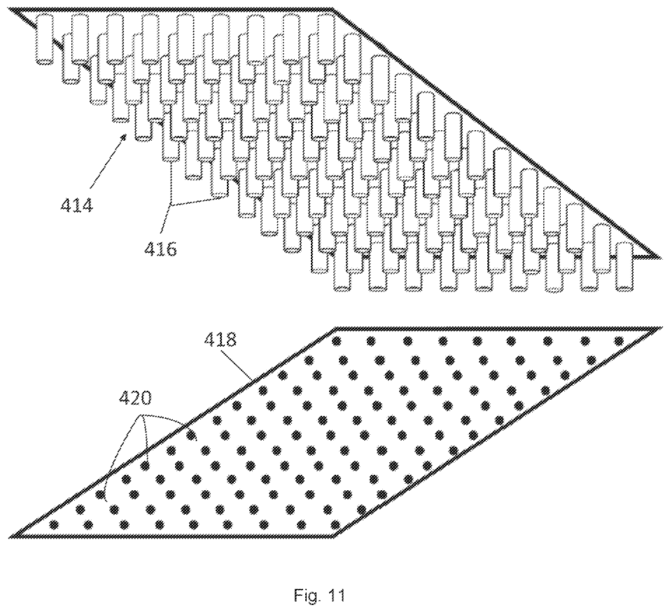

By way of exemplification and not limitation, FIG. 10 depicts a high level block diagram of a general purpose computer system suitable for use in performing functions described herein. As depicted in FIG. 10, system 400 comprises a processor 402, a memory 404, a storage device 406, and communicatively connected via a system bus 408. In some embodiments, processor 402 can comprise a microprocessor, central processing unit (CPU), or any other like hardware based processing unit. In some embodiments, a CMM 410 can be stored in memory 404, which can comprise random access memory (RAM), read only memory (ROM), optical read/write memory, cache memory, magnetic read/write memory, flash memory, or any other non-transitory computer readable medium. In some embodiments, processor 402 and memory 404 can be used to execute and manage the operation of CMM 410. In some embodiments, storage device 406 can comprise any storage medium or storage unit that is configured to store data accessible by processor 402 via system bus 408. Exemplary storage devices can comprise one or more local databases hosted by system 400. In some embodiments, a printer 412 is communicatively connected via a system bus 408 and is used to print reagents such as polynucleotides, using one or more toner cartridges TC, as disclosed herein. In some embodiments, system 400 can comprise a component 414 for hydrating or otherwise applying a reaction component such as buffer to printed coordinates 420. In some embodiments, component 414 comprises a pin array comprising pins 416. By way of elaboration and not limitation, and with reference to FIGS. 3A, 10 and 11, an assembly line type approach can be employed, wherein the substrate or paper 418 is laser printed/solid state deposition of reagents (at coordinates 420), and the print heads/toner catridges TC are lined up like an assembly line with printer 412. Then, the substrate/paper 418 rolls along through the printer 412 to a pin array/acoustic liquid deposition device 414, or like device hydrates coordinates 420 on the substrate/paper 418.

In some embodiments, component or pin array 414, could represent acoustic liquid handling, could represent microfluidic tubing, or could be metal pins (e.g, pins 416) that are dipped into a liquid to take up small drops and then stamped onto a substrate 420.

As indicated above, the subject matter described herein can be implemented in software in combination with hardware and/or firmware. For example, the subject matter described herein can be implemented in software executed by a processor. In one exemplary implementation, the subject matter described herein can be implemented using a computer readable medium having stored thereon computer executable instructions that, when executed by a processor of a computer, control the computer to perform steps. Exemplary computer readable mediums suitable for implementing the subject matter described herein include non-transitory devices, such as disk memory devices, chip memory devices, programmable logic devices, and application specific integrated circuits. In addition, a computer readable medium that implements the subject matter described herein can be located on a single device or computing platform or can be distributed across multiple devices or computing platforms. As used herein, the term “module” refers to hardware, firmware, or software in combination with hardware and/or firmware for implementing features described herein.

### REFERENCES

All references listed below, as well as all references cited in the instant disclosure, including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.
Kadkhodaei et al., RSC Adv., 2016,6, 66682-66694
US2018/0265921A1
CA2878042 A1
WO2014014991 A2
EP2875458 A2
US20050053968 A1
WO2013178801 A2
US20130019572 A1
WO2004088585 A2
WO2013178801 A2
US20150261664 A1
WO2015090879 A1
CA2874540 A1
EP2856375 A2
US20040001371 A1
WO2004088585 A2
U.S. Pat. No. 7,659,175
U.S. patent application Ser. No. 11/766,222
U.S. Pat. No. 9,262,738
WO2014014991
WO 2020/096679
U.S. Pat. No. 9,834,774
US 2019/0351673 AI
Gao et al, www.biorxiv.org/content/10.1101/2020.02.09.940411v3
Palluk et al., Nature Biotechnology Volume 36, pages 645-650 (2018) Service, www.sciencemag.org/news/2018/10/new-way-write-dna-could-turbocharge-synthetic-biology-and-data-storage
Lee et al., Nature Communications volume 10, Article number: 2383 (2019)
Troutt et al., PNAS Oct. 15, 1992 89 (20) 9823-9825

The following additional references correspond to footnote numbers presented in the specification.
1. Jonathan P. L. Cox. Long-term data storage in DNA. TRENDS Biotechnol. 19, 247-250 (2001).
2. Church, G. M., Gao, Y. & Kosuri, S. Next-Generation Digital Information Storage in DNA. Science 337, 1628 (2012).
3. Goldman, N. et al. Towards practical, high-capacity, low-maintenance information storage in synthesized DNA. Nature 494, 77-80 (2013).
4. Yazdi, S. M. H. T., Yuan, Y., Ma, J., Zhao, H. & Milenkovic, O. A Rewritable, Random-Access DNA-Based Storage System. Sci. Rep. 5, 14138 (2015).
5. Bornholt, J. et al. A DNA-based archival storage system. in Proceedings of the Twenty-First International Conference on Architectural Support for Programming Languages and Operating Systems-ASPLOS '16 337, 637-649 (2016).
6. Deng, L. et al. Optimized Code Design for Constrained DNA Data Storage with Asymmetric Errors. IEEE Access 1 (2019). doi:10.1109/ACCESS.2019.2924827
7. Haughton, D. & Balado, F. BioCode: two biologically compatible Algorithms for embedding data in non-coding and coding regions of DNA. BMC Bioinformatics 14, 121 (2013).
8. Lee, H. H., Kalhor, R., Goela, N., Bolot, J. & Church, G. M. Terminator-free template-independent enzymatic DNA synthesis for digital information storage. Nat. Commun. 10, 1-12 (2019).
9. Carlson, R. Synthesis. http://www.synthesis.cc/synthesis/2016/03/on_dna_and_transistors. (2016).
10. Doroschak, K. et al. Porcupine: Rapid and robust tagging of physical objects using nanopore-orthogonal DNA strands. bioRxiv 2020.03.06.981514 (2020). doi:10.1101/2020.03.06.981514
11. Erlich, Y. & Zielinski, D. DNA Fountain enables a robust and efficient storage architecture. Science 355, 950-954 (2017).
12. Extance, A. How DNA could store all the world's data. Nature 537, 22-24 (2016).
13. Takahashi, C. N., Nguyen, B. H., Strauss, K. & Ceze, L. Demonstration of End-to-End Automation of DNA Data Storage. (2018). doi:10.1101/439521
14. Organick, L. et al. Random access in large-scale DNA data storage. Nat Biotechnol 36, 242-248 (2018).
15. Tomek, K. J. et al. Driving the Scalability of DNA-Based Information Storage Systems. ACS Synth. Biol. 8, 1241-1248 (2019).
16. Lin, K. N., Volkel, K., Tuck, J. M. & Keung, A. J. Dynamic and scalable DNA-based information storage. Nat. Commun. 11, (2020).
17. Organick, L. et al. Probing the physical limits of reliable DNA data retrieval. Nat. Commun. 11, (2020).
18. Shankland, S. https://www.cnet.com/news/startup-packs-all-16gb-wikipedia-onto-dna-strands-demonstrate-new-storage-tech/. (2019).
19. Tomek, K. J., Volkel, K., Indermaur, E. W., Tuck, J. M. & Keung, A. J. Promiscuous molecules for smarter file operations in DNA-based data storage. Nat. Commun. 12, (2021).
20. TwistBioscience. Oligo Pools product information. www.twistbioscience.com DSOPUS_031218. (2018).
21. Caruthers, M. H. & McBride, L. J. An investigation of several deoxynucleoside phosphoramidites useful for synthesizing deoxyoligonucleotides. Tetrahedron Lett. (1983). doi:10.1017/CBO9781107415324.004
22. TwistBioscience. A Simple Guide to Phosphoramidite Chemistry and How it Fits in Twist Bioscience's Commercial Engine. https://www.twistbioscience.com/blog/science/simple-guide-phosphoramidite-chemistry-and-how-it-fits-twist-biosciences-commercial. (2018).
23. Sigma Aldrich. DNA Oligonucleotide Synthesis. https://www.sigmaaldrich.com/technical-documents/articles/biology/dna-oligonucleotide-synthesis.html.
24. Halleran, A. D., Swaminathan, A. & Murray, R. M. Single Day Construction of Multigene Circuits with 3G Assembly. ACS Synth. Biol. 7, 1477-1480 (2018).
25. An, Y. et al. A rapid and efficient method for multiple-site mutagenesis with a modified overlap extension PCR. Appl. Microbiol. Biotechnol. 68, 774-778 (2005).
26. Potapov, V. et al. Comprehensive Profiling of Four Base Overhang Ligation Fidelity by T4 DNA Ligase and Application to DNA Assembly. ACS Synth. Biol. 7, 2665-2674 (2018).
27. Takahashi, C. N., Nguyen, B. H., Strauss, K. & Ceze, L. Demonstration of End-to-End Automation of DNA Data Storage. Sci. Rep. 9, 1-5 (2019).
28. Roquet, N. et al. DNA-based data storage via combinatorial assembly. bioRxiv (2021). doi:https://doi.org/10.1101/2021.04.20.440194
29. LABCYTE. Echo® Liquid Handlers REDEFINE SCIENTIFIC INNOVATION WITH ACOUSTIC LIQUID HANDLING Echo®. www.labcyte.com/echo (2019).
30. Podivinsky, E., Love, J. L., Colff, L. van der & Samuel, L. Effect of storage regime on the stability of DNA used as a calibration standard for real-time polymerase chain reaction. Anal. Biochem. 394, 132-134 (2009).
31. Trapmann, S. et al. Development of a novel approach for the production of dried genomic DNA for use as standards for qualitative PCR testing of food-borne pathogens. Accredit. Qual. Assur. 9, 695-699 (2004).
32. Colotte, M., Coudy, D., Tuffet, S. & Bonnet, J. Adverse effect of air exposure on the stability of DNA stored at room temperature. Biopreserv. Biobank. 9,47-50 (2011).
33. Kohll, A. X. et al. Stabilizing synthetic DNA for long-Term data storage with earth alkaline salts. Chem. Commun. 56, 3613-3616 (2020).
34. Kwok, K. Y. et al. Strategies for maintaining the particle size of peptide DNA condensates following freeze-drying. Int. J. Pharm. 203, 81-88 (2000).
35. Newman, S. et al. High density DNA data storage library via dehydration with digital microfluidic retrieval. Nat. Commun. 10, 1-6 (2019).
36. Mlitz, K. Information archiving solutions market size worldwide 2014-2025. (2021).
37. Intelligence, M. ENTERPRISE INFORMATION ARCHIVING MARKET-GROWTH, TRENDS, COVID-19 IMPACT, AND FORECASTS (2021-2026). (2021).
38. Cui, M. & Zhang, Y. Incorporating Randomness into DNA Steganography to Realize Secondary Secret key, Self-destruction, and Quantum Key Distribution-like Function. bioRxiv 725499 (2019). doi:10.1101/725499
39. QStar. Long-term Data Archiving. (2021). Available at: https://www.qstar.com/document-archiving-best-practice/.
40. Iron Mountain. COST, SECURITY AND RELIABILITY LEAD DATA ARCHIVE REQUIREMENTS. (2021).
41. Micro Focus. The Six Key Drivers for Archiving. doi:https://www.microfocus.com/media/top-reasons/the_six_key_drivers_for_archiving_tr.pdf
42. Tongish, S. Archival Storage Trends and Challenges. Plasmon doi:https://williamsdatamanagement.com/wp-content/uploads/2017/04/archivaltrends_WP.pdf
43. Helixworks Technologies. Molecular Storage System (MoSS). https://helix.works
44. Roquet, N., Park, H. & Bhatia, S. P. Nucleic Acid-Based Data Storage. US 2018/0137418. http://www.freepatentsonline.com/y2018/0137418.html. (2018).
45. MarketsAndMarkets. Enterprise Information Archiving Market by Type (Content Types (Email, Database, Social Media, Instant Messaging, and Web) and Services (Consulting and System Integration)), Deployment Mode, Enterprise Size, and Region—Global Forecast to 2025. https://w. (2020).
46. Hoist, A. Information archiving solutions market size worldwide 2014-2024. Statista (2020).
47. Sverdlik, Y. There are Now More than 500 Hyperscale Data Centers in the World. Data Center Knowledge (2019).
48. Estimating Data Center Cost of Ownership: 5 Hidden Costs. https://www.datafoundry.com/blog/estimating-data-center-cost-ownership-5-hidden-costs. Data Foundry (2019).
49. Zhou, Z. et al. Fine-Grained Energy Consumption Model of Servers Based on Task Characteristics in Cloud Data Center. IEEE Access 6, 27080-27090 (2017).
50. Vidal, J. ‘Tsunami of data’ could consume one fifth of global electricity by 2025. Clim. Home News (2017).
51. McClelland, B. Designing a high performance tape archive. https://www.versity.com/blog/designing-a-high-performance-tape-archive?hs_amp=true&_twitter_impression=true. Versity Big Archive (2018).
52. Fujifilm. More than 10 Reasons to Buy Fujifilm Branded LTO Tape Drives. https://www.pmddatasolutions.com/admin/resources/more-than-10-reasons-to-buy-fujifilm-branded-1to-tapes.pdf. (2018).
53. Nordrum, A. Storing data in glass: Optical storage media is advancing from CDs and DVDs to small glass squares. IEEE Spectr. 57, 10-11 (2020).
54. Cafferty, B. J. et al. Storage of Information Using Small Organic Molecules. ACS Cent Sci 5, 911-916 (2019).
55. Kennedy, E. et al. Encoding information in synthetic metabolomes. PLoS One (2019). doi:10.1371/journal.pone.0217364
56. Rosenstein, J. K. et al. Principles of Information Storage in Small-Molecule Mixtures. IEEE Trans. Nanobioscience (2020). doi:10.1109/TNB.2020.2977304
57. Lee, J. M. et al. High-density information storage in an absolutely defined aperiodic sequence of monodisperse copolyester. Nat Commun 11, 56 (2020).
58. Gabrys, R., Pattabiraman, S. & Milenkovic, O. Mass Error-Correction Codes for Polymer-Based Data Storage. (2020).
59. Pattabiraman, S., Gabrys, R. & Milenkovic, O. Coding for Polymer-Based Data Storage. 1-20 (2020).
60. Palluk, S. et al. De novo DNA synthesis using polymerase-nucleotide conjugates. Nat Biotechnol 36, 645-650 (2018).
61. Gravitz, L. The Dawn of Cheap and Easy DNA Writing. https://neo.life/2019/11/the-dawn-of-cheap-and-easy-dna-writing/. Neo.Life (2019).
62. DNA Script Expands Series B to $89M. https://www.dnascript.com/press-releases/dna-script-expands-series-b-to-89m/. DNA Script (2020).
63. Papiewski, J. Comparison of Printer Speeds. https://smallbusiness.chron.com/comparison-printer-speeds-60220.html. Chron
64. Grass, R. N., Heckel, R., Puddu, M., Paunescu, D. & Stark, W. J. Robust chemical preservation of digital information on DNA in silica with error-correcting codes. Angew. Chemie-Int. Ed. 54, 2552-2555 (2015).
65. Bancroft, C. Long-Term Storage of Information in DNA. Science (80-.). 293, 1763c-1765 (2001).
66. Cox, J. P. Long-term data storage in DNA. Trends Biotechnol 19, 247-250 (2001).
67. Roquet, N. et al. PRINTER-FINISHER SYSTEM FOR DATA STORAGE IN DNA. US 2019/0351673. http://www.freepatentsonline.com/y2019/0351673.html. (2019).
68. Helixworks Technologies. Molecular Storage System(MoSS). https://helix.works.
69. Meece, K. A., Strack, C. D. & Tomson, T. D. S. METHOD AND APPARATUS FOR AUTOMATICALLY COMPENSATING A DEGRADATION OF THE CHARGE ROLLER VOLTAGE IN A LASER PRINTER. (2000).
70. Jiang, H. & Lu, L. Measurement of the surface charge of ultrafine particles from laser printers and analysis of their electrostatic force. Atmos. Environ. 44, 3347-3351 (2010).
71. Hoshino, Y., Kutsuwada, N., Watanabe, Y. & Izawa, H. Measurement of van der waals force of toner adhesion employing a linearly increasing electric field and determining the toner jumping voltage. Part. Sci. Technol. (1996). doi:10.1080/02726359608906700
72. Longo, P. M., Kavran, J. M., Kim, M.-S. & Leahy, D. J. Transient mammilian cell transfection with PEI. Methods Enzymol. (2012). doi:10.1016/j.biotechadv.2011.08.021.Secreted
73. Yang, Y. et al. The effect of amorphous calcium phosphate on protein protection against thermal denaturation. Chem. Commun. (2015). doi:10.1039/c5cc01420d
74. Denning, W. et al. Optimization of the transductional efficiency of lentiviral vectors: Effect of sera and polycations. Mol. Biotechnol. (2013). doi:10.1007/s12033-012-9528-5
75. Pardee, K. et al. Paper-based synthetic gene networks. Cell (2014). doi:10.1016/j.cell.2014.10.004
76. Takahashi, M. K. et al. A low-cost paper-based synthetic biology platform for analyzing gut microbiota and host biomarkers. Nat. Commun. (2018). doi:10.1038/s41467-018-05864-4
77. New England Biolabs. Data Sheet for T4 DNA Ligase. https://www.neb.com/-/media/catalog/datacards-or-manuals/m0202datasheet-lot1071207.pdf
78. New England Biolabs. Datasheet for Q5 High-Fidelity DNA Polymerase. https://www.neb.com/-/media/catalog/datacards-or-manuals/m0491datasheet-lot0041207.pdf
79. New England Biolabs. Buffer and Diluent Formulation Table. https://www.neb.com/tools-and-resources/selection-charts/buffer-and-diluent-formulation-table.
80. New England Biolabs. Activity of DNA Modifying Enzymes in CutSmart Buffer. https://www.neb.com/tools-and-resources/usage-guidelines/activity-of-dna-modifying-enzymes-in-cutsmart-buffer.
81. Koch, J. et al. A DNA-of-things storage architecture to create materials with embedded memory. Nat. Biotechnol. 38, 39-43 (2020).
82. Olorunniji, F. J. et al. Multipart DNA assembly using site-specific recombinases from the large serine integrase family. in Methods in Molecular Biology (2017). doi:10.1007/978-1-4939-7169-5_19
83. Fujifilm. Disk Storage vs. Tape Storage. http://www.tape-storage.net/en/storage_comparison/article_01/.
84. Amazon EFS. Amazon EFS Infrequent Access. https://aws.amazon.com/efs/features/infrequent-access/. (2020).
85. DNA Data Storage Alliance. PRESERVING OUR DIGITAL LEGACY: AN INTRODUCTION TO DNA DATA STORAGE. (2021).

While the systems and methods have been described herein in reference to specific aspects, features, and illustrative embodiments, it will be appreciated that the utility of the subject matter is not thus limited, but rather extends to and encompasses numerous other variations, modifications and alternative embodiments, as will suggest themselves to those of ordinary skill in the field of the present subject matter, based on the disclosure herein. Various combinations and sub-combinations of the structures and features described herein are contemplated and will be apparent to a skilled person having knowledge of this disclosure. Any of the various features and elements as disclosed herein can be combined with one or more other disclosed features and elements unless indicated to the contrary herein. Correspondingly, the subject matter as hereinafter claimed is intended to be broadly construed and interpreted, as including all such variations, modifications and alternative embodiments, within its scope and including equivalents of the claims.

What is claimed is:

1. A method for solid phase transfer of a reagent to a substrate, the method comprising:
(a) providing at least a first composition comprising a solid phase, wherein the solid phase comprises a first reagent; and
(b) dispensing a first sample from the first composition onto a first coordinate on a substrate, whereby the first reagent is transferred to the substrate from the solid phase.

2. The method of claim 1, wherein the solid phase comprises a powder, a bead or a combination thereof, wherein the first reagent is provided on the powder, the bead, or the combination thereof.

3. The method of claim 1, wherein the substrate comprises a charge and/or the substrate comprises a reagent pre-loaded at one or more coordinates on the substrate.

4. The method of claim 1, wherein the first composition comprises a positively charged moiety or a negatively charged moiety.

5. The method of claim 1, wherein the first reagent comprises a chemical entity selected from the group consisting of a polynucleotide, a polypeptide, a lipid, a small molecule organic chemical, a detectable moiety, an inorganic chemical entity, and a molecular hybrid of any of the foregoing groups, or comprises a cell.

6. The method of claim 5, wherein the polynucleotide component comprises a first nucleic acid oligomer block.

7. The method of claim 6, wherein the first oligomer block comprises a codeword.

8. The method of claim 1, wherein step (b) comprises providing a laser printer comprising at least a first toner cartridge for the first composition, wherein the laser printer is configured to dispense the first sample from the first toner cartridge onto the coordinate on the substrate.

9. The method of claim 1, comprising providing at least a second composition comprising a solid phase, wherein the solid phase comprises a second reagent; and dispensing a second sample from the second composition onto a coordinate on a substrate, wherein the coordinate is the first coordinate or is a second, different coordinate, whereby the second reagent is transferred to the substrate from the solid phase.

10. The method of claim 9, comprising dispensing the first sample from the first composition onto a coordinate on the substrate and dispensing the second sample from the second composition onto the coordinate on the substrate, such that the first and second reagent are collocated on the substrate.

11. The method of claim 9 or claim 10, comprising providing a laser printer comprising at least a first toner cartridge for the first composition and a second toner cartridge for the second composition, wherein the laser printer is configured to dispense the first sample from the first toner cartridge onto the first coordinate on the substrate and to dispense a second sample from the second toner cartridge onto the first or the second coordinate on the substrate.

12. The method of claim 10 or claim 11, comprising providing a condition necessary to cause an interaction between the first and second reagent, such as a reaction between the first and second reagent, such as a reaction to physically link the first and second reagent.

13. The method of claim 12, comprising dispensing a reaction mix onto the first coordinate on the substrate to provide a condition necessary to cause an interaction between the first and second reagent, such as a reaction between the first and second reagent, such as a reaction to physically link the first and second reagent.

14. The method of claim 13, wherein the reaction mix comprises a ligase, a polymerase, a recombinase, an exonuclease, a restriction endonuclease, a nickase, or any combination thereof.

15. The method of any one of claims 8 and 11-14, wherein the laser printer, or a system comprising the laser printer, comprises a component configured to provide a condition necessary to cause an interaction between the first and second reagent, such as a reaction between the first and second reagent, such as a reaction to physically link the first and second reagent.

16. The method of claim 15, wherein the component comprises a third toner cartridge, an incubator, pin array, or any combination thereof.

17. The method of any one of claims 9-16, wherein the solid phase comprises a powder, a bead or a combination thereof, wherein the first reagent and the second reagent are provided on the powder, the bead, or the combination thereof.

18. The method of any one of claims 9-17, wherein the substrate comprises a charge and/or the substrate comprises a reagent pre-loaded at one or more coordinates on the substrate.

19. The method of any one of claims 9-18, wherein the first reagent and the second reagent each comprise a positively charged moiety or a negatively charged moiety.

20. The method of any one of claims 9-19, wherein the first reagent and the second reagent can be the same or different, and each comprise a chemical entity selected from the group consisting of a polynucleotide, a polypeptide, a lipid, a small molecule organic chemical, a detectable moiety, an inorganic chemical entity, and a molecular hybrid of any of the foregoing groups, or a cell.

21. The method of claim 20, wherein the polynucleotide component comprises a first nucleic acid oligomer block and a second nucleic acid oligomer block.

22. The method of claim 21, wherein the first oligomer block and the second oligomer each comprise a codeword.

23. The method of any one of claims 1-22, further comprising providing one or more additional compositions, each additional composition comprising an additional reagent, wherein each of the additional reagent can independently be the same or different from the first or the second reagent;
dispensing a sample from each of the one or more additional compositions onto a coordinate on a substrate, wherein the coordinate is the first coordinate, is the second coordinate, or is one or more different coordinates, whereby the each of the additional reagents is transferred to the substrate from the solid phase.

24. The method of claim 23, comprising a sample from each of the one or more additional compositions onto a coordinate on the substrate, such that the first, second, and one or more additional reagents are collocated on the substrate; and
optionally providing a condition necessary to cause an interaction between the first, second, and one or more additional reagents, such as a reaction between the first, second, and one or more additional reagents, such as a reaction to physically link the first, second, and one or more additional reagents.

25. The method of claim 23 or claim 24, comprising providing a laser printer comprising one or more additional toner cartridges for the one or more additional compositions, wherein the laser printer is configured to dispense a sample from each of the one or more additional toner cartridges onto a coordinate on the substrate, optionally wherein the first, second, and one or more additional reagents are collocated on the substrate.

26. A system suitable for use in carrying out any of the processes set forth in claims 1-25.
